(19) United States
(12) Patent Application Publication
(10) Pub. No.: US 2026/0042809 A1
(43) Pub. Date: Feb. 12, 2026

(54) PROCESS FOR THE PREPARATION OF SEMAGLUTIDE

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Venkat Reddy GHOJALA, Hyderabad, Telangana (IN); Venkat Reddy MALLEPALLY, Hyderabad, Telangana (IN)

(73) Assignee: MSN LABORATORIES PRIVATE LIMITED, R&D CENTER, Hyderabad, Telangana (IN)

(21) Appl. No.: 18/294,556

(22) PCT Filed: Aug. 4, 2022
(86) PCT No.: PCT/IN2022/050702

§ 371 Date: Feb. 2, 2024

(30) Foreign Application Priority Data
Aug. 4, 2021 (IN) 202141035146

(51) Int. Cl. C07K 14/605

(52) U.S. Cl. CPC C07K 14/605

(57) ABSTRACT

The present invention relates to an improved process for the preparation of Semaglutide. The present further relates to improved processes for the purification of various intermediate compounds of Semaglutide and their use in the preparation of pure Semaglutide. The present invention further relates to improved process for the purification of Semaglutide.

### RELATED APPLICATION

This application claims the benefit of priority of our Indian Patent Application number 202141035146 filed on Aug. 4, 2021 which is incorporated herein by reference.

### FIELD OF THE INVENTION

The present invention provides a process for the preparation of Semaglutide represented by the following structural formula-1.

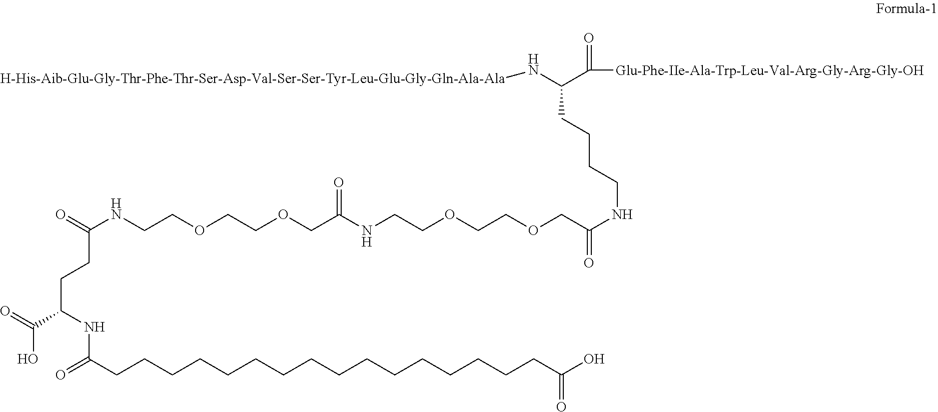

### BACKGROUND OF THE INVENTION

Semaglutide is a long-acting GLP-1 analog developed by Novo Nordisk. Semaglutide was approved in United States on Dec. 5, 2017, in Europe on Feb. 12, 2018 and it is marketed under the brand name OZEMPIC. It is a glucagon-like peptide 1 (GLP-1) receptor agonist indicated as an adjunct to diet and exercise to improve glycemic control in adults with type 2 diabetes mellitus.

U.S. Pat. No. 8,129,343 B2 and U.S. Pat. No. 8,536,122 B2 describes Semaglutide, its analogs and process for preparation thereof. The said process involves the preparation of Semaglutide backbone using standard sequential Fmoc solid phase peptide synthesis followed by deprotection and then coupling of the side chain fragment to the Lys20. The said route of synthesis has several disadvantages. For example, the sequential synthesis described results in low purity. Additionally, the coupling of the side chain moiety to Lys is carried out by using 17-((S)-1-tert-butoxycarbonyl-3-{2-[2-({2-[2-(2,5-dioxopyrrolidin-1-yloxycarbonylmethoxy)ethoxy]ethylcarbamoyl}methoxy)ethoxy]ethylcarbamoyl}propylcarbamoyl) heptadecanoic acid tert-butyl ester. In this process, due to the presence of tert-butyl protecting group on the side chain, an extra deprotection step is required. This results in the need of additional purification cycles and loss of overall yield. The said patents further disclose the usage of Dde as a protecting group on the Lys. This strategy is disadvantageous because hydrazine, which is a toxic and dangerous reagent, is required for the removal of the Dde protecting group.

Various prior art documents described the synthesis of Semaglutide.

WO2016046753A1 described the synthesis of Semaglutide by Fmoc solid phase synthesis by using Wang resin. Usage of Wang resin leads to the formation of para hydroxyl benzyl ester impurity.

WO2017114191A1 described the synthesis of Semaglutide which involves the usage of Fmoc-Lys (Alloc)-OH. Pd (PPh3) 4 is used for the removal of Alloc protecting group, which is not particularly suitable for the industrial scale synthesis of Semaglutide as the Pd (PPh3) 4 reagent is very sensitive to air, light and heat and also it is very expensive. Moreover Pd is defined as a highly toxic metal impurity and its presence in drug product must be minimized to significantly lower levels.

CN105753964A described the synthesis of Semaglutide by using Dde protected Lysine. The removal of Dde protecting group requires the usage of hydrazine as a deprotecting agent. Hydrazine is active in nature and risky in storage and transportation and unsafe in the process of large scale production.

Synthesis of Semaglutide by coupling of side chain with the peptide backbone in a linear or fragment approach during the backbone building lead to the formation of various PEG impurities. This impacts the final product quality and yield.

Semaglutide synthesized by a linear/sequential synthesis as described in the prior art documents results in low purity. On the other hand, the purification of Semaglutide is difficult due to its longer amino acids chain. Moreover, the impurities in the final product are difficult to remove.

Hence, in view of all the disadvantages, prior art processes are not viable for the preparation of Semaglutide on industrial scale.

Hence, there is a significant need to develop an improved process for the preparation and purification of Semaglutide.

The present inventors after numerous trails and earnest efforts developed a process for the preparation of Semaglutide which involves less number of synthetic steps, simple operations and easy to carry out chemical conversions. Further, a purification method has been developed by the present inventors which results in high pure Semaglutide in higher yield and can be useful on the industrial scale.

The process described in the present invention is simple, safe, economic and suitable for the production of Semaglutide and its side chain on industrial scale in good yield and better quality.

### BRIEF DESCRIPTION OF THE INVENTION

An embodiment of the present invention is to provide an improved process for the preparation of Semaglutide.

The other embodiment of the present invention is to provide an improved process for the preparation of compound of formula-2.

Another embodiment of the present invention is to provide an improved process for the preparation of compound of formula-3

Another embodiment of the present invention is to provide a process for the purification of Semaglutide backbone.

The other embodiment of the present invention is to provide a process for the purification of Semaglutide.

Another embodiment of the present invention is to provide a process for the purification of compound of formula-13.

Another embodiment of the present invention is to provide a process for the preparation of compound of formula-8.

Another embodiment of the present invention is to provide a process for the preparation of compound of formula-10.

### DETAILED DESCRIPTION OF THE INVENTION

The “solvent” used in the present invention can be selected from but not limited to “hydrocarbon solvents” such as n-pentane, n-hexane, n-heptane, cyclohexane, petroleum ether, benzene, toluene, xylene and the like; “ether solvents” such as dimethyl ether, diethyl ether, diisopropyl ether (DIPE), methyl tert-butyl ether (MTBE), 1,2-dimethoxyethane, tetrahydrofuran (THF), 2-methyltetrahydrofuran (2-MeTHF), 2-methoxyethyl ether (Diglyme), 1,4-dioxane and the like; “ester solvents” such as methyl acetate, ethyl acetate (EtOAc), n-propyl acetate, isopropyl acetate, n-butyl acetate, isobutyl acetate, tert-butyl acetate and the like; “polar-aprotic solvents” such as dimethylacetamide (DMAc), dimethylformamide (DMF), dimethylsulfoxide (DMSO), N-methylpyrrolidone (NMP) and the like; “chloro solvents” such as dichloromethane (DCM), dichloroethane, chloroform, carbon tetrachloride and the like; “ketone solvents” such as acetone, methyl ethyl ketone (MEK), methyl isobutyl ketone (MIBK) and the like; “nitrile solvents” such as acetonitrile (ACN), propionitrile, isobutyronitrile and the like; “alcohol solvents” such as methanol, ethanol, n-propanol, iso-propanol or isopropyl alcohol (IPA), n-butanol, iso-butanol, 2-butanol, tert-butanol, ethane-1,2-diol, propane-1,2-diol and the like; water; formic acid, acetic acid (AcOH) and the like or mixture of any of the afore mentioned solvents.

The “base” used in the present invention can be selected from but not limited to “inorganic bases” selected from “alkali metal carbonates” such as sodium carbonate, potassium carbonate, lithium carbonate, cesium carbonate and the like; “alkali metal bicarbonates” such as sodium bicarbonate, potassium bicarbonate, lithium bicarbonate, cesium bicarbonate and the like; “alkali metal hydroxides” such as sodium hydroxide, potassium hydroxide, lithium hydroxide, cesium hydroxide and the like; “alkali metal hydrides” such as sodium hydride, potassium hydride, lithium hydride and the like; “alkali metal amides” such as sodium amide, potassium amide, lithium amide and the like; ammonia; “organic bases” like “alkali metal alkoxides” such as sodium methoxide, sodium ethoxide, potassium methoxide, potassium ethoxide, lithium methoxide, lithium ethoxide, sodium tert.butoxide, potassium tert.butoxide, lithium tert.butoxide and the like; alkali metal and alkali earth metal salts of acetic acid such as sodium acetate, potassium acetate, magnesium acetate, calcium acetate and the like; dimethylamine, diethylamine, diisopropyl mine, diisopropylethylamine (DIPEA), diisobutylamine, trimethylamine, triethylamine (TEA), triisopropylamine, tributylamine, tert.butyl amine, pyridine, piperidine, 4-dimethylamino pyridine (DMAP), quinoline, imidazole, N-methylimidazole, 1,8-diazabicyclo[5.4.0]undec-7-ene (DBU), 1,5-diazabicyclo[4.3.0]non-5-ene (DBN), dimethylaniline, N-methylmorpholine (NMM), 1,4-diazabicyclo[2.2.2]octane (DABCO), 2,6-lutidine and the like; “organolithium bases” such as methyl lithium, n-butyl lithium (n-BuLi), lithium diisopropylamide (LDA) and the like; “organosilicon bases” such as lithium hexamethyldisilazide (LiHMDS), sodium hexamethyldisilazide (NaHMDS), potassium hexamethyldisilazide (KHMDS) and the like or mixtures thereof.

The “acid” in the present invention can be selected from but not limited to “inorganic acids” selected from hydrochloric acid, hydrobromic acid, sulfuric acid, nitric acid, phosphoric acid, boric acid, perchloric acid; “organic acids” selected from tartaric acid, acetic acid, maleic acid, citric acid, malic acid, oxalic acid, formic acid, trifluoroacetic acid.

The “coupling agent” used in the present invention can be selected from but not limited to N,N′-dicyclohexylcarbodiimide (DCC), N,N″-diisopropyl carbodiimide (DIC), 1-ethyl-3-(3-dimethylaminopropyl) carbodiimide hydrochloride (EDC.HCl), N,N″-carbonyl diimidazole (CDI), 1-[bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphate (HBTU), 1H-benzotriazolium 1-[bis(dimethylamino)methylene]-5-chloro-hexafluorophosphate (1)-3-oxide (HCTU), (benzotriazol-1-yloxy) tris(dimethylamino)phosphonium hexafluorophosphate (BOP), benzotriazol-1-yl-oxytripyrrolidinophosphonium hexafluorophosphate (PyBOP), 1-hydroxy-7-azatriazole (HOAt), 1-hydroxy benzotriazole (HOBt), 1-hydroxy-1H-1,2,3-triazole-4-carboxylate (HOCt),O-(benzotriazol-1-yl)-N,N,N′,N′-tetramethyluronium tetrafluoroborate (TBTU), N-hydroxysuccinamide (HOSu), N-hydroxysulfosuccinimide (Sulfo-NHS), ethyl cyanohydroxyiminoacetate (Oxyma), 7-Azabenzotriazol-1-yloxy)tripyrrolidinophosphonium hexafluorophosphate (PyAOP), N,N,N′,N′-Tetramethyl-O-(N-succinimidyl) uronium tetrafluoroborate (TSTU) or mixtures thereof.

The deprotection of Fmoc group in the present invention is carried out by using a base selected from piperidine, piperazine, DBU optionally in presence of a solvent such as DMF, NMP and the like.

The buffer solution used in the present invention is selected from but not limited to aqueous ammonia, aqueous ammonium chloride, aqueous ammonium acetate, aqueous ammonium bicarbonate, aqueous sodium acetate, aqueous sodium chloride, aqueous sodium carbonate, aqueous sodium bicarbonate, aqueous monosodium phosphate (NaH₂PO₄), aqueous disodium (Na₂HPO₄), phosphate aqueous KH₂PO₄, aqueous tris(hydroxymethyl)aminomethane (Tris buffer), aqueous formic acid, aqueous acetic acid, aqueous trifluoroacetic acid, citric acid in water, aqueous boric acid, aqueous sulfuric acid, isopropyl methylphosphonic acid (IMPA) in water, O-phthalaldehyde (OPA) in water or mixtures thereof.

The pH of the buffer solution in the present invention is about 1 to about 10. In one embodiment, the pH is about 1.5 to about 9. In one embodiment, the pH is about 2 to about 8. In one embodiment, the pH is about 2.5 to about 7. In one embodiment, the pH is about 3 to about 6. In one embodiment, the pH is about 3.5 to about 5. In one embodiment, the pH is about 4 to about 4.5.

In one embodiment of the present invention, the pH of the buffer solution is adjusted by using aqueous NaOH solution.

The molar concentration of the buffer solution in the present invention is about 1.5M to 0.01M.

In one embodiment, the buffer concentration is about 1.2 M to about 0.05 M. In one embodiment, the buffer concentration is about 1.0 M to about 0.07 M. In one embodiment, the buffer concentration is about 0.5 M to about 0.1 M.

The “cocktail mixture/cleaving reagent” in the present invention is used for cleavage of the peptide chain from the Resin or for the deprotection of amino acids or for both of them simultaneously.

The “cocktail mixture/cleaving reagent” in the present invention is selected from but not limited to HF, TFA (trifluoroacetic acid), TIS or TIPS (triisopropyl silane), Phenol, water, Anisole, Thioanisole, EDT (Ethane-1,2-dithiol), 1-dodecanethiol (DDT), Dithiothreitol (DTT), methanesulfonic acid or mixtures thereof.

In one embodiment, the “cocktail mixture/cleaving reagent” is selected from TFA:TIS:Phenol:water (8.25:0.5:1:0.25), TFA:TIS:Phenol:water (9.25:0.25:0.25:0.25), TFA:HF:Phenol:water (8.25:0.5:1:0.25), TFA:TIS:Anisole:water (8.25:0.5:1:0.25), TFA:TIS:Anisole:water (9.25:0.25:0.25:0.25), TFA:phenol:water:thioanisole:EDT (82.5:5:5:5:2.5), TFA:phenol:water:thioanisole: 1-dodecanethiol (82.5:5:5:5:2.5), TFA:DTT:water:TIPS (88:5:5:2), TFA:phenol:Methanesulfonicacid (95:2.5:2.5), TFA:thioanisole:EDT:anisole (90:5:3:2), TFA:TIS:Thioanisole:water (8.25:0.5:1:0.25), TFA:phenol:water:thioanisole:DTT (82.5:5:5:5:2.5) and the like.

The “diluent” in the present invention is selected from but not limited to methanol:water, ethanol:water, acetonitrile:water, isopropanol:water and the like.

The first embodiment of the present invention provides an improved process for the preparation of Semaglutide, comprising reacting (3-31) amino acid fragment of Semaglutide having the amino acid sequence Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly with PG₁-His (PG)-Aib-OH to provide a peptide having amino acid sequence His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly and optionally converting the obtained compound to Semaglutide.

In the above process, ‘PG₁’ represents a protecting group selected from alkyloxy carbonyl such as methoxy carbonyl, ethoxy carbonyl, tert.butoxycarbonyl (Boc) and the like; benzyloxycarbonyl (Cbz), 9-fluorenylmethyloxy carbonyl (FMOC), acetyl (Ac), benzoyl (Bz), benzyl (Bn), allyloxy carbonyl (Alloc), trityl (Trt).

‘PG’ represents a protecting group selected from trityl (Trt), tert.butyl (t-Bu), Tosyl (Tos), monomethoxy trityl (Mmt), methyltrityl (Mtt), tert.butoxycarbonyl (Boc), 2,4-dimethylpent-3-yloxycarbonyl (Doc), benzyloxymethyl (Bom), tert-butoxymethyl (Bum).

Various amino acids of (3-31) fragment of Semaglutide as described above are optionally protected with different protecting groups which include but not limited to alkyloxy carbonyl such as methoxy carbonyl, ethoxy carbonyl, tert.butoxycarbonyl (Boc) and the like; benzyloxycarbonyl (Cbz), 9-fluorenylmethyloxy carbonyl (FMOC), acetyl (Ac), benzoyl (Bz), benzyl (Bn), allyloxy carbonyl (Alloc), trityl (Trt), tert.butyl (tBu), 2,2,4,6,7-pentamethyl-2,3-dihydrobenzofuran-5-sulfonyl (Pbf), Tosyl (Tos), monomethoxy trityl (Mmt), methyltrityl (Mtt), 2,4-dimethylpent-3-yloxycarbonyl (Doc), benzyloxymethyl (Bom), tert-butoxymethyl (Bum) and the like.

In the first aspect of the first embodiment of the present invention, the (3-31) amino acid fragment of Semaglutide in the above process is bound to a solid support (Resin). For example, the solid support is Chlorotrityl chloride (CTC or 2-CTC) Resin or Wang resin.

In the second aspect of the first embodiment of the present invention, the (3-31) amino acid fragment of Semaglutide is synthesized by solid phase peptide synthesis (SPPS).

In the third aspect of the first embodiment of the present invention, the (3-31) amino acid fragment of Semaglutide is synthesized by solution phase peptide synthesis.

In the fourth aspect of the first embodiment of the present invention, the synthesis of (3-31) amino acid fragment of Semaglutide is carried out by combination of solid phase synthesis (SPPS) and solution phase synthesis.

In the fifth aspect of the first embodiment of the present invention, the (3-31) amino acid fragment of Semaglutide is prepared by a recombinant process.

In the sixth aspect of the first embodiment of the present invention, the solid phase synthesis or solution phase synthesis of (3-31) amino acid fragment of Semaglutide is carried out by linear approach by sequential coupling of 3-31 amino acids according to the amino acid sequence of Semaglutide backbone.

In the seventh aspect of the first embodiment of the present invention, the solid phase synthesis or solution phase synthesis of (3-31) amino acid fragment of Semaglutide is carried out by fragment approach.

In an aspect of the present invention, the (3-31) amino acid fragment of Semaglutide has the formula Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-Resin and the amino acids of the sequence are optionally protected with protecting group(s).

In an aspect of the present invention, peptide having the amino acid sequence His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly has the formula His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-Resin wherein the amino acids of the sequence are optionally protected with protecting group(s).

The above described process is carried out in a solvent optionally in presence of a coupling agent and/or a base.

The solvent is selected from hydrocarbon solvents, ether solvents, ester solvents, polar-aprotic solvents, chloro solvents, ketone solvents, nitrile solvents, water and the like or mixtures thereof.

The coupling agent is selected from those described above and the base wherever necessary is selected from organic bases.

In one aspect of the first embodiment of the present invention, the conversion of His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-Resin (wherein the amino acids of the sequence are optionally protected with protecting group(s)) to Semaglutide is carried out by cleavage of the peptide chain from the Resin and deprotection of all the amino acids wherever applicable by using “cocktail mixture/cleaving reagent” and reacting the obtained compound with compound of formula-3 in a solvent optionally in presence of a base and/or a coupling agent.

In the other aspect of the first embodiment of the present invention, the conversion is carried out by deprotection of Lysine (if applicable), coupling of compound of formula-3 to the backbone at Lysine followed by deprotection of the amino acids and cleavage of the peptide chain from the Resin by using “cocktail mixture/cleaving reagent”.

Various protected and unprotected amino acids, PG₁-His (PG)-Aib-OH used in the above described process can be added as a single lot or lots wise to the reaction mixture.

The second embodiment of the present invention provides a process for the preparation of Semaglutide, comprising;
a) reacting Boc-His (PG)-Aib-OH with H-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin to provide Boc-His (PG)-Aib-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin (Resin bound Semaglutide backbone),
b) converting the compound obtained in step a) to Semaglutide.

The reaction in step a) is carried out in a solvent optionally in presence of a base and/or a coupling agent which are selected from those defined above.

In the first aspect of the second embodiment of the present invention, the conversion in step b) is carried out by cleavage of the peptide chain from the Resin and global deprotection of the amino acids followed by reacting the obtained compound with compound of formula-3.

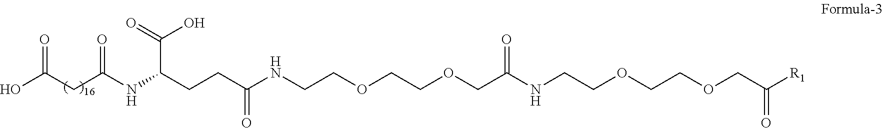

Wherein, ‘R₁’ represents substituted or unsubstituted aryloxy and the substituents wherever necessary can be independently selected from halogens such as F, Cl, Br & I, NO₂ and the substitution can takes place at single or multiple positions on aryl group or R₁ is

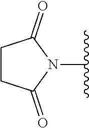 [SMILES: *N1C(=O)CCC1=O]

In the second aspect of the second embodiment of the present invention, the conversion is carried out by deprotection of Lysine at position 20, coupling of compound of formula-3 to the backbone at Lysine followed by global deprotection and cleavage of the peptide chain from the Resin.

The present invention further provides a process for the preparation of Semaglutide, comprising reacting H-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-OH (Semaglutide backbone) with compound of formula-3. The reaction is carried out in presence of a solvent optionally in presence of a base and/or a coupling agent.

The present inventors when carried out the reaction of (3-31) amino acid fragment of Semaglutide having the amino acid sequence Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly with PG₁-His (PG)-Aib-OH and further converted the obtained compound to Semaglutide surprisingly found that the said process efficiently controlled the formation of D-His, Des-His, Des-Aib, Di-Aib, Des-Aib-His impurities in the Semaglutide API which is highly advantageous on industrial scale especially for complex peptide molecules like Semaglutide.

The third embodiment of the present invention provides a process for the preparation of H-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin, comprising;
a) Coupling of Fmoc-Gly-OH with Resin in a solvent and a base by solid phase peptide synthesis to provide Fmoc-Gly-Resin,
b) treating Fmoc-Gly-Resin with a deprotecting agent to provide H-Gly-Resin,
c) coupling of Fmoc-Arg (pbf)-OH to H-Gly-Resin in a solvent optionally in presence of a coupling agent and/or a base to provide Fmoc-Arg (Pbf)-Gly-Resin,
d) treating Fmoc-Arg (Pbf)-Gly-Resin with a deprotecting agent to provide H-Arg (Pbf)-Gly-Resin,
e) repeating the coupling and deprotection steps with remaining amino acids Fmoc-Gly-OH, Fmoc-Arg (pbf)-OH, Fmoc-Val-OH, Fmoc-Leu-OH, Fmoc-Trp (Boc)-OH, Fmoc-Ala-OH, Fmoc-Ile-OH, Fmoc-Phe-OH, Fmoc-Glu (OtBu)-OH, Fmoc-Lys (Boc)-OH, Fmoc-Ala-OH, Fmoc-Ala-OH, Fmoc-Gln (Trt)-OH, Fmoc-Gly-OH, Fmoc-Glu (OtBu)-OH, Fmoc-Leu-OH, Fmoc-Tyr (tBu)-OH, Fmoc-Ser (tBu)-OH, Fmoc-Ser (tBu)-OH, Fmoc-Val-OH, Fmoc-Asp (OtBu)-OH, Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH by linear approach as per the amino acid sequence in Semaglutide backbone in a solvent optionally in presence of a coupling agent and/or a base.

The solvent in step a) to step e) is selected from hydrocarbon solvents, ether solvents, ester solvents, polar-aprotic solvents, chloro solvents, ketone solvents, nitrile solvents, water and the like or mixture of any of the afore mentioned solvents.

The base in step a), step c) and step e) is selected from those described above.

The deprotecting agent for Fmoc deprotection is selected from bases such as piperidine, piperazine, DBU optionally in presence of a solvent such as DMF, NMP and the like. The coupling agent in step a), step c) and step e) is selected from those described above.

In the above process, ‘PG’ is selected from those as defined above.

The fourth embodiment of the present invention provides an improved process for the preparation of compound of formula-2 (Boc-His (PG)-Aib-OH), comprising;

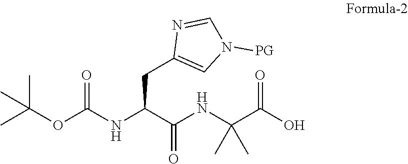

wherein, ‘PG’ represents protecting group as defined above;
a) treating compound of formula-4 with R—OH optionally in presence of a catalyst and/or a solvent to provide compound of formula-5,

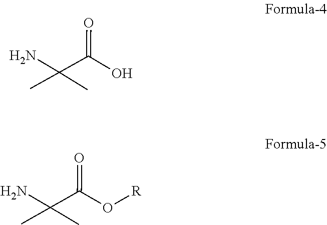

wherein, ‘R’ represents C₁-C₆ straight chain or branched chain alkyl group;
b) reacting compound of formula-5 with compound of formula 6,

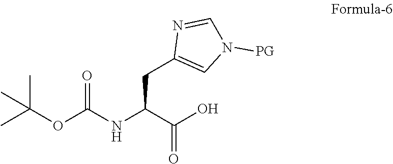

in a solvent optionally in presence of a coupling agent and/or a base to provide compound of formula-7,

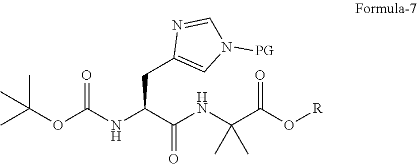

c) hydrolysis of compound of formula-7 in presence of an acid or a base optionally in presence of a solvent to provide compound of formula-2.

The solvent in step-a) to step-c) is selected from hydrocarbon solvents, ether solvents, ester solvents, polar-aprotic solvents, chloro solvents, ketone solvents, nitrile solvents, water and the like or mixture of any of the afore mentioned solvents.

The catalyst in step-a) is selected from thionyl chloride, oxalyl chloride, methanesulfonyl chloride, phosphorus trichloride, phosphorus pentachloride, phosphoryl chloride, acids such as HCl, H₂SO₄, methanesulfonic acid and the like.

The base in step-b) is selected from inorganic bases, organic bases or mixture thereof.

The coupling agent in step-b) is selected from those defined above.

The acid in step-c) is selected from inorganic acids and the base is selected from inorganic bases.

The fifth embodiment of the present invention provides a process for the purification of compound of formula-2 having compound of formula-6 (Histidine impurity) as an impurity at higher levels (Histidine impurity >1%), comprising purifying the compound of formula-2 from a solvent.

The solvent for the above purification process is selected from ether solvents such as dimethyl ether, diethyl ether, diisopropyl ether, methyl tert-butyl ether, 1,2-dimethoxyethane, tetrahydrofuran, 2-methyl tetrahydrofuran, 2-methoxyethyl ether (Diglyme), 1,4-dioxane and the like; chloro solvents such as dichloromethane, dichloroethane, chloroform, carbon tetrachloride and the like or mixtures thereof.

Various techniques are used for purification of compound of formula-2 viz., recrystallization, addition of anti-solvent to a solution of formula-2 in a solvent, and the like.

The compound of formula-2 used as an input for the above purification process can be prepared as per the process described in the present invention or by any of the processes known in the art.

In an aspect, the compound of formula-2 (input material) in the present invention is having compound of formula-6 as an impurity at a level of about 10%, or about 5%, or about 4%, or about 3%, or about 2%, or about 1%, or about 0.5% as measured by HPLC.

The sixth embodiment of the present invention provides an improved process for the preparation of compound of formula-3 (Semaglutide side chain), comprising;
a) reacting compound of formula-8 with compound of formula-10 in a solvent optionally in presence of a coupling agent and/or a base to provide compound of formula-11,
b) reacting compound of formula-11 with compound of formula-10 in a solvent optionally in presence of a coupling agent and/or a base to provide compound of formula-13,
c) optionally purifying compound of formula-13,
d) treating compound of formula-13 with compound of formula-14

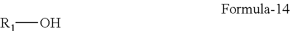

wherein, ‘R₁’ is as defined above;
in a solvent optionally in presence of a coupling agent and/or a base to provide compound of formula-15,

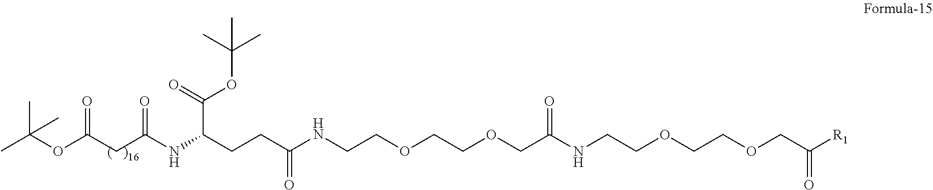

e) treating compound of formula-15 with a deprotecting agent optionally in presence of a solvent to provide compound of formula-3.

Coupling agent in step-a), step-b) & step-d) is selected from those described above.

The base in step-a), step-b) & step-d) is selected from inorganic bases, organic bases or mixtures thereof.

The solvent in step-a) to step-e) wherever necessary is selected from hydrocarbon solvents, ether solvents, ester solvents, polar-aprotic solvents, chloro solvents, ketone solvents, nitrile solvents, water and the like or mixtures thereof.

The deprotecting agent in step-e) is selected from acids as described above and inorganic bases.

The seventh embodiment of the present invention provides a process for the preparation of compound of formula-3a (Semaglutide side chain), comprising:
a) preparation of compound of formula-13 as per the process described above,
b) reacting compound of formula-13 with compound of formula-14a in a solvent optionally in presence of a coupling agent and/or a base to provide compound of formula-15a

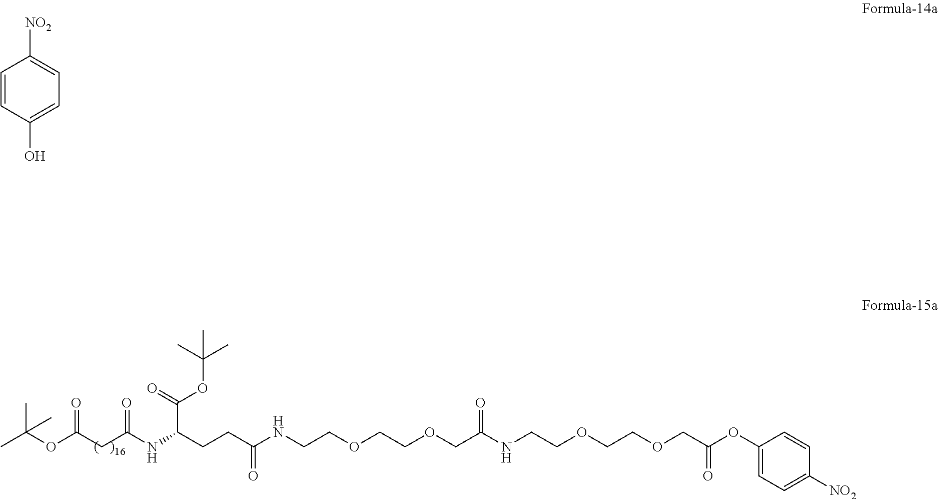

c) treating compound of formula-15a with a deprotecting agent optionally in presence of a solvent to provide compound of formula-3a.

The coupling agent in step-b) is selected from those described above.

The base in step-b) is selected from inorganic bases, organic bases or mixtures thereof.

The solvent in step-b) to step-c) wherever necessary is selected from hydrocarbon solvents, ether solvents, ester solvents, polar-aprotic solvents, chloro solvents, ketone solvents, nitrile solvents, water and the like or mixtures thereof.

The deprotecting agent in step-c) is selected from acids as described above and inorganic bases.

The eighth embodiment of the present invention provides a process for the purification of Semaglutide backbone comprising, subjecting a solution of Semaglutide backbone to RP-HPLC (Reverse phase high performance liquid chromatography) purification by using acetonitrile optionally in mixture with alcohol solvent as mobile phase.

The alcohol solvent in the above purification process is selected from methanol, ethanol and isopropyl alcohol.

In the above process, solution of Semaglutide backbone is prepared by dissolving Semaglutide backbone in buffer solution.

The ninth embodiment of the present invention provides a process for the purification of Semaglutide backbone comprising, one or more of the following steps;
a) dissolving crude Semaglutide backbone in a buffer solution,
b) subjecting the solution to RP-HPLC purification using buffer solution as mobile phase A and acetonitrile optionally in mixture with alcohol solvent as mobile phase B,
c) subjecting the solution to RP-HPLC purification by using buffer solution as mobile phase A and acetonitrile as mobile phase B,
d) subjecting the solution to RP-HPLC by using water as mobile phase A and acetonitrile as mobile phase B.

In the above purification process, buffer solution is selected from those described above; and the alcohol solvent is selected from C₁-C₆ straight chain or branched chain alcohols such as methanol, ethanol, n-propyl alcohol, isopropyl alcohol, n-butyl alcohol, iso-butyl alcohol, tert-butyl alcohol, n-pentanol and the like.

In step-d) of the above described process in place of water, aqueous TFA can be used as mobile phase A and the pH of the aqueous TFA solution can be adjusted by using aqueous NaOH solution. In an aspect, the pH is adjusted to 4-5 by using aqueous NaOH solution.

The tenth embodiment of the present invention provides a process for the purification of Semaglutide comprising, subjecting a solution of Semaglutide to RP-HPLC purification by using acetonitrile optionally in mixture with alcohol solvent as mobile phase.

In the above process, solution of Semaglutide is prepared by dissolving Semaglutide in buffer solution.

The eleventh embodiment of the present invention provides a process for the purification of Semaglutide, comprising one or more of the following steps;
a) dissolving Semaglutide in a buffer solution,
b) subjecting the solution to RP-HPLC purification by using buffer solution as mobile phase A and acetonitrile optionally in mixture with alcohol solvent as mobile phase B,
c) subjecting the solution to RP-HPLC by using buffer solution as mobile phase A and acetonitrile as mobile phase B.

In the above purification process, the buffer solution and the alcohol solvent are selected from those described in ninth embodiment of the present invention.

The twelfth embodiment of the present invention provides a process for the purification of Semaglutide by RP-HPLC, comprising 0.025M Ammonium acetate in water as mobile phase A and Acetonitrile:Isopropyl alcohol as mobile phase B.

The present invention provides a process for the purification of Semaglutide by RP-HPLC comprising aqueous H₂SO₄ as mobile phase. This process further comprising acetonitrile optionally in mixture with aqueous H₂SO₄ as mobile phase B.

The thirteenth embodiment of the present invention provides a process for the purification of Semaglutide by RP-HPLC comprising 0.025% H₂SO₄ in water as mobile phase A and Acetonitrile:Methanol as mobile phase B.

An embodiment of the present invention provides a process for the purification of Semaglutide by RP-HPLC comprising aqueous H₂SO₄ as mobile phase A and Acetonitrile:aqueous H₂SO₄ as mobile phase B.

Other embodiment of present invention provides process for purification of Semaglutide by RP-HPLC comprising water as mobile phase A and acetonitrile as mobile phase B.

Semaglutide which is used as input in the purification processes of the present invention can be prepared as per the process described in the present invention or by any other processes described in the art.

The fourteenth embodiment of the present invention provides a process for the purification of compound of forumula-13, comprising subjecting a solution of compound of formula-13 to RP-HPLC purification by using acetonitrile as mobile phase.

The solution of compound of formula-13 in the above described process is obtained by dissolving the compound in diluent. The diluent is described from those described above.

The above described process for the purification of compound of formula-13 further comprising treating obtained fractions with an acid. In an aspect, the acid is inorganic acid.

An embodiment of present invention provides a process for purification of compound of forumula-13, comprising subjecting a solution of compound of formula-13 to RP-HPLC purification by using buffer solution as mobile phase-A and acetonitrile as mobile phase-B.

In the above described process, the buffer solution is prepared by adjusting the pH of the water to 3-4 with aqueous TFA solution.

The compound of formula-8 which is used in the above described process is prepared by a process, comprising,
a) treating compound of formula-16 with di.tert-butyl dicarbonate (DIBOC), t-butanol in presence of a base in a solvent to provide compound of formula-17,
b) reacting compound of formula-17 with compound of formula-19 in a solvent optionally in presence of a coupling agent and/or a base to provide compound of formula-8.

The base in step-a) and step-b) is selected from inorganic bases, organic bases or mixtures thereof.

The solvent in step-a) and step-b) wherever necessary is selected from hydrocarbon solvents, ether solvents, ester solvents, polar-aprotic solvents, chloro solvents, ketone solvents, nitrile solvents, water and the like or mixtures thereof.

The coupling agent in step-b) is selected from those defined above.

The compound of formula-10 which is used in the present invention can be prepared by a process comprising:
a) treating compound of formula-20 with compound of formula-21 in presence of a solvent and/or a base optionally in presence of an alkali metal halide to provide compound of formula-22,

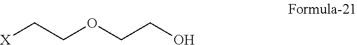

wherein, ‘X’ represents halogens such as Cl, Br and I;
b) reacting compound of formula-22 with compound of formula-23 in a solvent optionally in presence of a base to provide compound of formula-24,

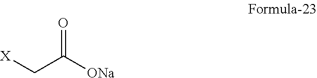

c) debenzylating the compound of formula-24 with a debenzylating agent in presence of a solvent to provide the compound of formula-10,
d) optionally purifying the compound of formula-10 with a solvent to provide pure compound of formula-10.

The alkali metal halide in step-a) is selected from potassium fluoride, potassium chloride, potassium bromide, potassium iodide, sodium fluoride, sodium chloride, sodium bromide, sodium iodide, lithium fluoride, lithium chloride, lithium bromide, lithium iodide and the like.

The solvent in step-a) to step-d) wherever necessary is selected from hydrocarbon solvents, ether solvents, ester solvents, polar-aprotic solvents, chloro solvents, ketone solvents, nitrile solvents, water and the like or mixtures thereof.

The base in step-a) and step-b) is selected from inorganic bases, organic bases or mixtures thereof.

The debenzylating agent in step-c) is selected from Pd/C, Raney-Ni, Pd (OH) 2/C, palladium acetate, platinum oxide, Rhodium and the like.

In one aspect of the above described process, the debenzoylation step can be carried out as a single step or two steps. i.e., compound of formula-24 can be treated with a debenzylating agent to provide mono benzyl compound which is further treated with a debenzylating agent to provide compound of formula-10.

Different grades of stationary phase can be used in the purification processes of the present invention which includes but not limited to C18 columns, C8 columns, C4 columns, phenyl columns, polymeric adsorbent columns, RP-amide columns and the like.

The flow rate in the RP-HPLC purification processes of the present invention is about 0.1 mL/min, or about 0.2 mL/min, or about 0.3 mL/min, or about 0.4 mL/min, or about 0.5 mL/min, or about 0.6 mL/min, or about 0.7 mL/min, or about 0.8 mL/min, or about 0.9 mL/min, or about 1 mL/min, or about 2 mL/min, or about 3 mL/min, or about 4 mL/min, or about 5 mL/min, or about 10 mL/min, or about 15 mL/min, or about 20 mL/min, or about 25 mL/min, or about 30 mL/min, or about 40 mL/min, or about 50 mL/min, or about 60 mL/min, or about 70 mL/min, or about 80 mL/min, or about 90 mL/min, or about 100 mL/min, or about 110 mL/min, or about 120 mL/min, or about 130 mL/min, or about 140 mL/min, or about 150 mL/min, or about 160 mL/min, or about 170 mL/min, or about 180 mL/min, or about 190 mL/min, or about 200 mL/min.

The run time in the RP-HPLC purification processes of the present invention is about 50 min, or about 60 min, or about 70 min, or about 80 min, or about 90 min, or about 100 min, or about 110 min, or about 120 min, or about 140 min, or about 160 min, or about 180 min, or about 200 min, or about 220 min, or about 240 min, or about 260 min, or about 280 min, or about 300 min, or about 320 min, or about 340 min, or about 360 min or about 380 min or about 400 min.

The wavelength is about 200 nm, or about 205 nm, or about 210 nm, or about 215 nm, or about 220 nm, or about 225 nm, or about 230 nm, or about 235 nm, or about 240 nm, or about 245 nm or about 250 nm.

Semaglutide obtained by the process of the present invention is having purity of greater than 90% by HPLC. In one embodiment, Semaglutide is having purity of greater than 95% by HPLC. In one embodiment, Semaglutide is having purity of greater than 96% by HPLC. In one embodiment, Semaglutide is having purity of greater than 97% by HPLC. In one embodiment, Semaglutide is having purity of greater than 98% by HPLC. In one embodiment, Semaglutide is having purity of greater than 98.5% by HPLC. In one embodiment, Semaglutide is having purity of greater than 98.8% by HPLC. In one embodiment, Semaglutide is having purity of greater than 99% by HPLC. In one embodiment, Semaglutide is having purity of greater than 99.2% by HPLC. In one embodiment, Semaglutide is having purity of greater than 99.5% by HPLC.

Semaglutide backbone obtained by the process of the present invention is having purity of greater than 90% by HPLC. In one embodiment, Semaglutide backbone is having purity of greater than 95% by HPLC. In one embodiment, Semaglutide backbone is having purity of greater than 96% by HPLC. In one embodiment, Semaglutide backbone is having purity of greater than 97% by HPLC. In one embodiment, Semaglutide backbone is having purity of greater than 98% by HPLC. In one embodiment, Semaglutide backbone is having purity of greater than 98.5% by HPLC. In one embodiment, Semaglutide backbone is having purity of greater than 98.8% by HPLC. In one embodiment, Semaglutide backbone is having purity of greater than 99% by HPLC.

The compound of formula-13 obtained by the process of the present invention is having purity of greater than 90% by HPLC. In one embodiment, compound of formula-13 is having purity of greater than 95% by HPLC. In one embodiment, compound of formula-13 is having purity of greater than 96% by HPLC. In one embodiment, compound of formula-13 is having purity of greater than 97% by HPLC. In one embodiment, compound of formula-13 is having purity of greater than 98% by HPLC. In one embodiment, compound of formula-13 is having purity of greater than 99% by HPLC.

### HPLC Method of Analysis:

Semaglutide: Column: C18 column; Wavelength: 220 nm; Diluent: 10 mm Na₂HPO₄ (with diluted Orthophosphoric acid); Elution:Gradient; Buffer:Sulfuric acid in water. Mobile phase-A: Buffer (100%) v/v; Mobile phase-B: Methanol: Acetonitrile: Sulfuric acid.
Semaglutide backbone: Column: C18 column; Wavelength: 225 nm; Diluent: Ammonia solution; Elution: Gradient; Buffer: Trifluoroacetic acid in water. Mobile phase-A: Buffer (100%) v/v; Mobile phase-B: Methanol: Acetonitrile.
Compound of formula-2: Column: C18 column; Wavelength: 220 nm; Diluent: Methanol; Elution: Gradient; Buffer: Sulfuric acid in water; Mobile phase-A: Buffer (100%) v/v; Mobile phase-B: Acetonitrile: Methanol.
Compound of formula-8: Column: RP-Amide column; Wavelength: 220 nm; Diluent: Methanol: Water (80:20) v/v; Elution: Gradient; Buffer: Perchloric acid in water; Mobile phase-A: Buffer (100%) v/v; Mobile phase-B: Acetonitrile: Methanol.
Compound of formula-13: Column: C18 column; Wavelength: 220 nm; Diluent: Methanol: Water (80:20) v/v; Elution: Gradient; Buffer: Perchloric acid in water; Mobile phase-A: Buffer (100%) v/v; Mobile phase-B: Acetonitrile: MeOH.
Semaglutide backbone: Column: C18 column; Wavelength: 225 nm; Diluent: 0.1% Ammonia solution; Elution: Gradient; Buffer: Sulphuric acid in water. Mobile phase-A: Buffer (100%) v/v; Mobile phase-B: Acetonitrile: Water (90:10) v/v.
Semaglutide: Column: C18 column; Wavelength: 220 nm; Diluent: Water; Elution: Gradient; Buffer: Sulphuric acid in water. Mobile phase-A: Buffer (100%) v/v; Mobile phase-B: Acetonitrile: Methanol: Sulfuric acid (50:50:0.05) v/v/v.
Compound of formula-13: Column: C18 column; Wavelength: 220 nm; Diluent: Methanol: Water (80:20) v/v; Elution: Gradient; Buffer: Perchloric acid in water; Mobile phase-A: Buffer (100%) v/v; Mobile phase-B: Acetonitrile: MeOH (75:25) v/v.

Semaglutide obtained as per the process of the present invention is useful for the preparation of various pharmaceutical compositions formulated in a manner suitable for the route of administration to be used.

Semaglutide produced according to present invention is useful for formulating into various dosage forms which include oral solids (for example tablets, capsules), oral liquids (powder for suspension, syrup, solution, emulsion), injections and the like.

The fifteenth embodiment of the present invention provides the use of Semaglutide obtained as per the process of the present invention for the preparation of pharmaceutical formulations.

The sixteenth embodiment of the present invention provides a pharmaceutical composition comprising Semaglutide of the present invention and at least one pharmaceutically acceptable excipient.

The seventeenth embodiment of the present invention provides a method of treating or preventing a disease comprising administering to the patient a therapeutically effective amount of Semaglutide obtained as per the process of the present invention.

The present invention is schematically represented as follows;

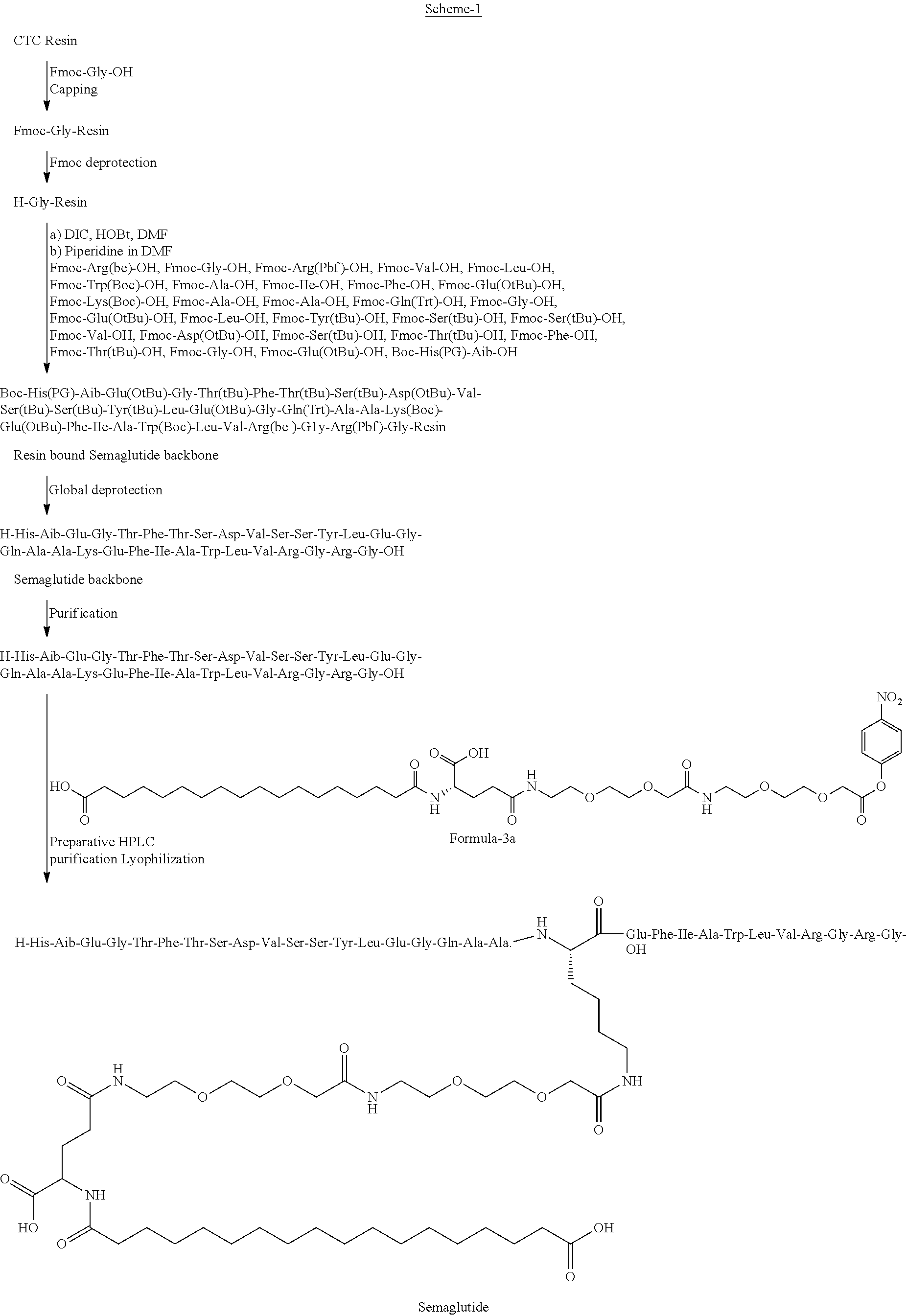

Wherein, ‘PG’ is a protecting group selected from trityl (Trt), tert.butyl (t-Bu), Tosyl (Tos), monomethoxy trityl (Mmt), methyltrityl (Mit), tert.butoxycarbonyl (Boc), 2,4-dimethylpent-3-yloxycarbonyl (Doc), benzyloxymethyl (Bom), tert-butoxymethyl (Bum).

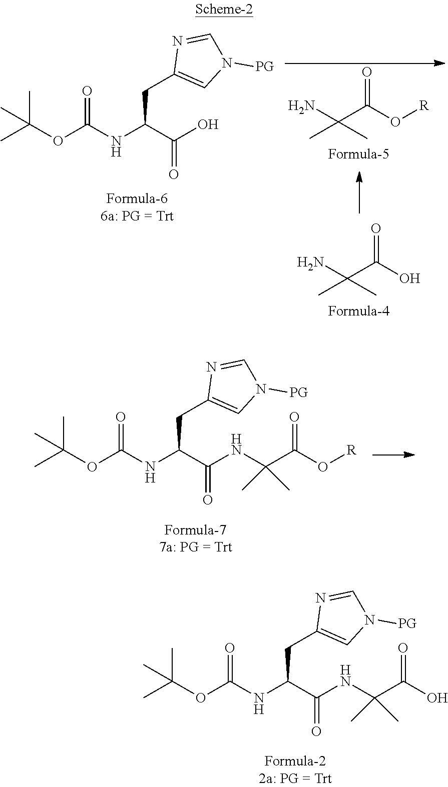

“PG” is a protecting group selected from those described above and ‘R’ represents C₁-C₆ alkyl group.

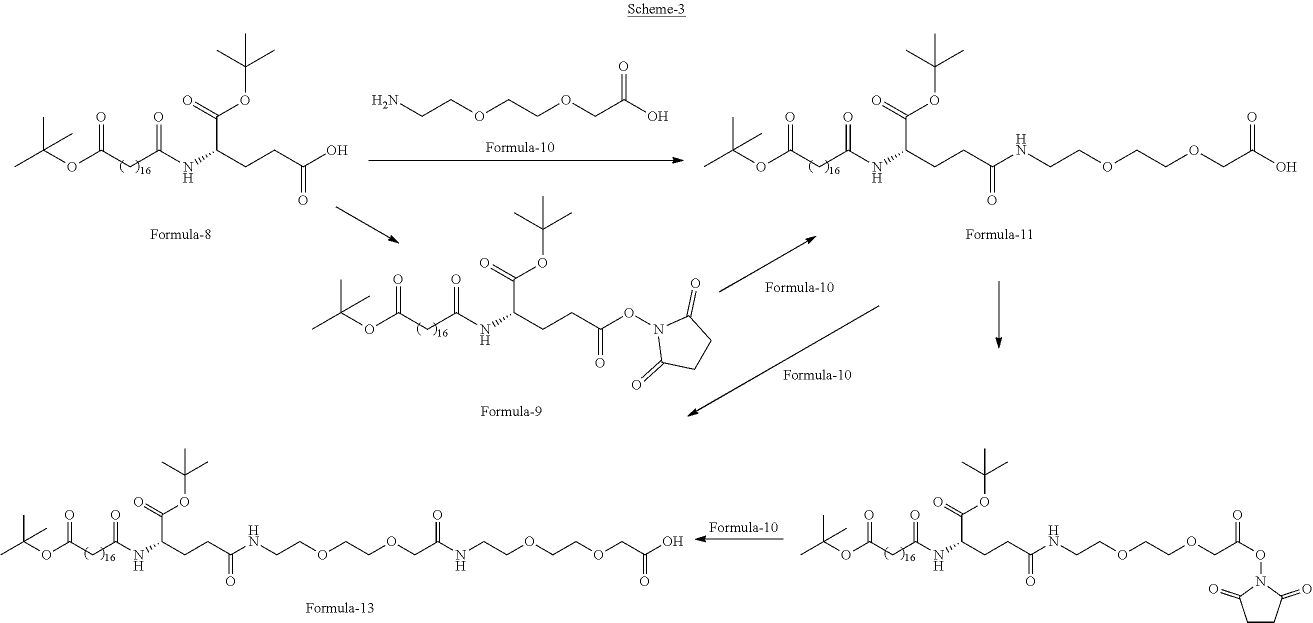

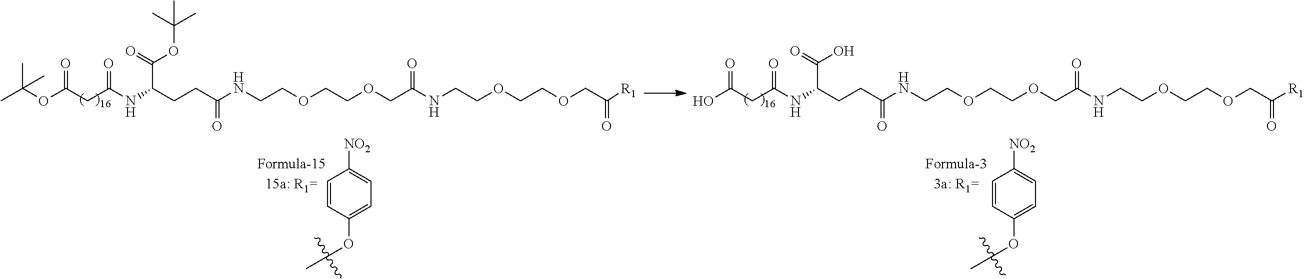

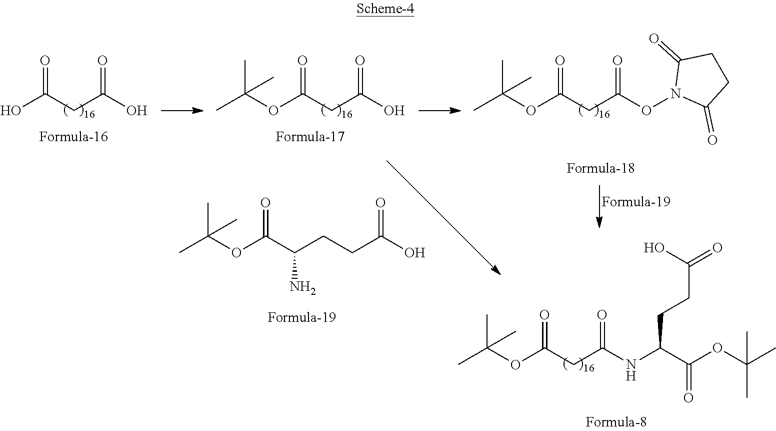

The best mode of carrying out the present invention is illustrated by the below mentioned examples. These examples are provided as illustration only and hence should not be construed as limitation to the scope of the invention.

### EXAMPLES

### Example—1: Preparation of Resin Bound Semaglutide Backbone

### Step 1: Preparation of Fmoc-Gly-CTC Resin

A solution of Fmoc-Gly-OH (19.02 gm) in DCM (250 ml) and DIPEA (13.93 ml) was added to CTC Resin (25 gm; swelled in DCM) at 25-30° C. and stirred the mixture for 6 hr at the same temperature. Drained out the solvent from the peptide flask and washed the reaction mixture with DMF. Capped the resin by using DCM (250 ml), methanol (25 ml) and DIPEA (6.96 ml) at 25-30° C. and stirred the mixture for 1 hr at the same temperature. Drained out the solvent from the peptide flask and washed the reaction mixture with DMF to get the title compound.

### Step 2: Deprotection of Fmoc-Gly-CTC Resin

20% Piperidine solution (50 ml of piperidine in 200 ml of DMF) was added to Fmoc-Gly-CTC Resin obtained in step 1 at 25-30° C. and stirred the reaction mixture for 40 min at the same temperature. Drained out the solvent from the peptide flask and washed the reaction mixture with DMF followed by DCM to get H-Gly-CTC Resin.

Step 3: Preparation of H-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin (3-31)

Fmoc-Arg (Pbf)-OH (37.7 gm) was dissolved in DMF (125 ml) at 25-30° C. HOBt (7.85 gm) and DIC (9.09 ml) were added to the mixture at 25-30° C. and stirred the mixture for 15 min at the same temperature. The resulting solution was added to H-Gly-CTC Resin obtained in step 2 at 25-30° C. and stirred the reaction mixture for 4 hr at the same temperature. Drained out the solvent from the peptide flask and washed the reaction mixture with DMF to get Fmoc-Arg (Pbf)-Gly-CTC Resin. 20% Piperidine solution (50 ml of piperidine in 200 ml of DMF) was added to the obtained reaction mixture at 25-30° C. and stirred for 30 min at the same temperature. Drained out the solvent from the peptide flask and washed the reaction mixture with DMF to get H-Arg (Pbf)-Gly-CTC Resin.

Repeated the above amino acids coupling and Fmoc deprotection steps with remaining amino acids as per the amino acid sequence of Semaglutide by using Fmoc-Gly-OH, Fmoc-Arg (pbf)-OH, Fmoc-Val-OH, Fmoc-Leu-OH, Fmoc-Trp (Boc)-OH, Fmoc-Ala-OH, Fmoc-Ile-OH, Fmoc-Phe-OH, Fmoc-Glu (OtBu)-OH, Fmoc-Lys (Boc)-OH, Fmoc-Ala-OH, Fmoc-Ala-OH, Fmoc-Gln (Trt)-OH, Fmoc-Gly-OH, Fmoc-Glu (OtBu)-OH, Fmoc-Leu-OH, Fmoc-Tyr (tBu)-OH, Fmoc-Ser (tBu)-OH, Fmoc-Ser (tBu)-OH, Fmoc-Val-OH, Fmoc-Asp (OtBu)-OH, Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH coupled to H-Arg (Pbf)-Gly-CTC resin by linear approach to get the title compound.

### Step 4: Preparation of Resin Bound Semaglutide Backbone

Boc-His (Trt)-Aib-OH compound of formula-2 (40.63 gm) was dissolved in DMF (250 ml) at 25-30° C. and HOBt (9.42 gm) and DIC (10.91 ml) were added to the reaction mixture at the same temperature. This obtained mixture was slowly added lot wise to the compound obtained in step-3 at 25-30° C. and stirred for 9 hr at the same temperature. Drained out the solvent from the peptide flask and washed with DMF followed by methanol and dried to get the title compound. Yield: 74.0 gm.

### Example—2: Preparation of Resin Bound Semaglutide Backbone

Step 1: Preparation of H-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin (3-31)

Fmoc-Arg (Pbf)-OH (42.57 gm) was dissolved in DMF (125 ml) at 25-30° C. and HOBt (8.86 gm) and DIC (10.12 ml) were added to it. The obtained reaction mixture was added to H-Gly-CTC Resin (obtained in similar manner to example 1) at 25-30° C. and stirred for 4 hr at the same temperature. Drained out the solvent from the peptide flask and washed the reaction mixture with DMF to get Fmoc-Arg (Pbf)-Gly-CTC Resin.

20% Piperidine solution (50 ml of piperidine in 200 ml of DMF) was added to the above obtained reaction mixture at 25-30° C. and stirred for 40 min at the same temperature. Drained out the solvent from the peptide flask and washed the reaction mixture with DMF to get H-Arg (Pbf)-Gly-CTC Resin.

Repeated the above amino acids coupling and Fmoc deprotection steps with remaining amino acids as per the amino acid sequence of Semaglutide by using Fmoc-Gly-OH, Fmoc-Arg (pbf)-OH, Fmoc-Val-OH, Fmoc-Leu-OH, Fmoc-Trp (Boc)-OH, Fmoc-Ala-OH, Fmoc-Ile-OH, Fmoc-Phe-OH, Fmoc-Glu (OtBu)-OH, Fmoc-Lys (Boc)-OH, Fmoc-Ala-OH, Fmoc-Ala-OH, Fmoc-Gln (Trt)-OH, Fmoc-Gly-OH, Fmoc-Glu (OtBu)-OH, Fmoc-Leu-OH, Fmoc-Tyr (tBu)-OH, Fmoc-Ser (tBu)-OH, Fmoc-Ser (tBu)-OH, Fmoc-Val-OH, Fmoc-Asp (OtBu)-OH, Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH coupled to H-Arg (Pbf)-Gly-CTC Resin by linear approach to provide the title compound.

### Step 2: Preparation of Resin Bound Semaglutide Backbone (1-31)

Boc-His (Trt)-Aib-OH compound of formula-2 (46.16 gm) was dissolved in DMF (250 ml) at 25-30° C. HOBt (10.53 gm) and DIC (12.14 ml) were added to the obtained solution at 25-30° C. This obtained mixture was slowly added lot wise to the compound obtained in step-1 at 25-30° C. and stirred for 9 hr at the same temperature. Drained out the solvent from the peptide flask and washed with DMF followed by methanol and dried to get the title compound. Yield: 72.0 gm.

### Example—3: Cleavage and Global Deprotection to Prepare Semaglutide Backbone

Resin bound Semaglutide backbone (120 gm) was slowly added to a pre-cooled mixture of TFA (990 ml), TIS (60 ml), water (30 ml) and phenol (120 ml) at 0-5° C. and stirred the reaction mixture for 15 min at the same temperature. Raised the temperature of the reaction mixture to 25-30° C. and stirred for 4 hr at the same temperature. Filtered the reaction mixture and washed with TFA. The obtained filtrate was slowly added to pre-cooled MTBE (3.6 Lt) at 0-5° C. and stirred the reaction mixture for 30 min at the same temperature. Raised the temperature of the reaction mixture to 25-30° C. and stirred for 3 hr at the same temperature. Filtered the solid, washed with MTBE and suck dried. MTBE (1.2 Lt) was added to the obtained compound at 25-30° C. and stirred the mixture for 2 hr at the same temperature. Filtered the solid, washed with MTBE and dried to get the title compound. Yield: 66 gm; Purity by HPLC: 56.05%.

### Example—4: Purification of Semaglutide Backbone by RP-HPLC

Step 1: The crude Semaglutide backbone (16 gm) was dissolved in 800 ml of aqueous ammonia solution and sonicated. The solution was filtered through 0.45μ filter paper. The C18 column was washed with Acetonitrile: Water and then equilibrated with aqueous Ammonium acetate+aqueous Ammonia. The crude solution was loaded onto the column and the column was stabilized with aqueous Ammonium acetate+aqueous Ammonia. A purification cycle was performed under the following conditions;
Mobile Phase A: Aqueous Ammonium acetate+Aqueous Ammonia; Mobile Phase B: Acetonitrile: Methanol; Elution: Gradient.
The desired fractions were collected and distilled off the solvent under reduced pressure to obtain an aqueous solution containing Semaglutide backbone. Purity by HPLC: 90.15%.
Step 2: The solution obtained in step 1 was loaded on to C18 HPLC column and the column was equilibrated with aqueous TFA. A purification cycle was performed by using Mobile Phase A: Aqueous TFA and Mobile Phase B: Acetonitrile. The desired fractions were collected and distilled off the solvent from the fractions under reduced pressure to obtain an aqueous solution containing Semaglutide backbone. Purity by HPLC: 99.02%.
Step 3: The C18 column was washed with Acetonitrile: Water and then equilibrated with aqueous ammonium acetate. The solution obtained in step 2 was loaded on to the column and stabilized the column with aqueous ammonium acetate. The column was subjected to gradient program by using water as mobile Phase A and acetonitrile as mobile Phase B. Desired fractions were collected and distilled off the solvent from the fractions to obtain an aqueous solution containing Semaglutide backbone. The obtained solution was lyophilized to get pure Semaglutide backbone as a solid. Yield: 2.4 gm; HPLC Purity: 98.63%.

### Example—5: Purification of Semaglutide Backbone by RP-HPLC

Step 1: The crude Semaglutide backbone (16 gm) was dissolved in 800 ml of aqueous ammonia solution and sonicated. The solution was filtered through 0.45μ filter paper. The C18 column was washed with Acetonitrile: Water and then equilibrated with aqueous Ammonium acetate+aqueous ammonia. The crude solution was loaded onto the column and the column was stabilized with aqueous Ammonium acetate+aqueous Ammonia. A purification cycle was performed under the following conditions;
Mobile Phase A: aqueous Ammonium acetate+aqueous Ammonia; Mobile Phase B: Acetonitrile: Methanol; Elution: Gradient.
The desired fractions were collected and distilled off the solvent under reduced pressure to obtain an aqueous solution containing Semaglutide backbone. Purity by HPLC: 89.7%.
Step 2: 50% of the solution obtained in step 1 was subjected to RP-HPLC purification as per the process described in step 2 of example 4. The desired fractions were collected and distilled off the solvent from the fractions under reduced pressure to obtain an aqueous solution containing Semaglutide backbone. Purity by HPLC: 99.04%.
Step 3: C18 column was washed with Acetonitrile: Water and then equilibrated with aqueous ammonium acetate. The solution obtained in step 2 was loaded on to column and stabilized the column with aqueous ammonium acetate. The column was subjected to gradient program by using Mobile Phase A: Water and Mobile Phase B: Acetonitrile. Desired fractions were collected and distilled off the solvent from the fractions under reduced pressure to obtain aqueous solution containing Semaglutide backbone. The obtained solution was lyophilized to get pure Semaglutide backbone as a solid. Yield: 1.9 gm; HPLC Purity: 98.65%.

### Example—6: Preparation of Semaglutide

Water (200 ml) was added to purified Semaglutide backbone (4 gm) at 25-30° C. Cooled the mixture to 15-20° C., DIPEA (2.05 ml) was added to it and stirred the mixture for 15 min at the same temperature. A solution of compound of formula-3a (1.6 gm) in THF (178 ml) was slowly added lot wise to the reaction mixture at 15-20° C. and stirred for 3 hr at same temperature. DIPEA (1.02 ml) was added to the reaction mixture at 15-20° C. and stirred for 2 hr at the same temperature. A solution of compound of formula-3a (0.2 gm) in THF (22 ml) was added to the reaction mixture at 15-20° C. and stirred the reaction mixture for 4 hr at the same temperature. Distilled off tetrahydrofuran from the reaction mixture under reduced pressure. Ethyl acetate was added to the reaction mixture at 25-30° C. and stirred for 30 min at the same temperature. Both the organic and aqueous layers were separated and NaCl was added to the aqueous layer. Cooled the reaction mixture to 0-5° C., slowly acidified with aqueous HCl solution and stirred for 24 hr at the same temperature. Filtered the compound, acetonitrile (16 ml) and methyl tert.butyl ether (64 ml) were added and stirred the mixture for 15 min at the same temperature. Filtered the compound, methyl tert.butyl ether (80 ml) was added to it at 25-30° C. and stirred for 15 min at same temperature. Filtered the solid and dried. Acetonitrile (40 ml) and acetone (40 ml) were added to the obtained compound at 25-30° C. and stirred the mixture for 2 hr at the same temperature. Filtered the solid, washed with acetone and dried to get the title compound. Yield: 4.42 gm; Purity by HPLC: 90.59%.

### Example—7: Purification of Semaglutide by RP-HPLC

Step 1: The crude Semaglutide (4 gm) was dissolved in aqueous ammonia solution and sonicated. The C18 column was equilibrated with aqueous Ammonium acetate. The crude solution was loaded on to the column and the column was stabilized with aqueous Ammonium acetate. A purification cycle was performed by using the following conditions; Mobile Phase A: Aqueous ammonium acetate; Mobile Phase B: Acetonitrile: Isopropyl alcohol; Elution: Gradient.
The desired fractions were collected and distilled off the solvent under reduced pressure to obtain an aqueous solution containing Semaglutide. Purity by HPLC: 98.89%.
Step 2: The C18 column was equilibrated with Aetonitrile: Water followed by washed with aq. NaHCO₃. Column was stabilized with water. The solution obtained in step 1 was loaded on the column and washed with aqueous NaHCO₃ solution. The column was eluted with gradient program by using Mobile Phase A: Water and Mobile Phase B: Acetonitrile.
The desired fractions were collected and distilled off the solvent under reduced pressure to obtain an aqueous solution containing Semaglutide. The obtained solution was subjected to lyophilization to get pure Semaglutide as a solid. Yield: 2.0 gm; HPLC Purity: 98.78%.

### Example—8: Process for the Purification of Semaglutide by RP-HPLC

Step 1: The crude Semaglutide (6 gm) was dissolved in aqueous ammonia solution and sonicated. The C18 column was equilibrated with dilute H₂SO₄. The crude solution was loaded onto the C18 column and the column was stabilized with mobile phase-A. The purification cycle was performed under the following conditions:
Mobile Phase A: Dil.H₂SO₄; Mobile Phase B: Acetonitrile: Methanol; Elution: Gradient
The desired fractions were collected and distilled off the solvent from the fractions to obtain an aqueous solution containing Semaglutide. Purity by HPLC: 97.36%.
Step 2: The C18 column was equilibrated with Acetonitrile: Water followed by washed with aqueous sodium bicarbonate solution. Column was stabilized with water. The solution obtained in step 1 was loaded on the column and washed the column with aqueous NaHCO₃ solution. The column was eluted with gradient program by using Mobile Phase A: Water and Mobile Phase B: Acetonitrile.
The desired fractions were collected and distilled off the solvent under reduced pressure to obtain an aqueous solution containing Semaglutide. The obtained solution was subjected to lyophilization to get pure Semaglutide as a solid. Yield: 2.6 gm; HPLC Purity: 98.71%.

### Example—9: Preparation of Compound of Formula-2

### Step 1: Preparation of Compound of Formula-5a

Thionyl chloride (17.6 ml) was slowly added to a mixture of ethanol (102 ml) and 2-aminoisobutyric acid compound of formula-4 (10.2 gm) at 25-30° C. and stirred the reaction mixture for 20 min at the same temperature. Heated the reaction mixture to 70-75° C. and stirred for 9 hr at the same temperature. Distilled off the solvent from the reaction mixture under reduced pressure and co-distilled with ethyl acetate. Ethyl acetate (102 ml) was added to the obtained compound at 25-30° C. and stirred the mixture for 1 hr 30 min at the same temperature. Filtered the solid and washed with ethyl acetate to get the title compound.

### Step 2: Preparation of Compound of Formula-7a

EDC.HCl (17.45 gm) and HOBt (8.14 gm) were added to a pre-cooled mixture of compound obtained in step-1 and tetrahydrofuran (150 ml) at 0-5° C. DIPEA (10.5 ml) followed by Boc-His (Trt)-OH compound of formula-6 (30 gm) were added to the reaction mixture at 0-5° C. DIPEA (10.5 ml) was slowly added to the reaction mixture at 0-5° C. and stirred for 30 min at the same temperature. Raised the temperature of the reaction mixture to 25-30° C. and stirred for 9 hr at the same temperature. Cooled the reaction mixture to 0-5° C., water (300 ml) was slowly added to it and stirred for 15 min at the same temperature. Raised the temperature of the reaction mixture to 25-30° C. and stirred for 2 hr at the same temperature. Filtered the solid, washed with water and dried to get the title compound.

### Step 3: Preparation of Compound of Formula-2

THF (210 ml) was added to the compound obtained in step-2 at 25-30° C. Aqueous LiOH solution (3.6 gm of LiOH in 90 ml of water) was added to the reaction mixture at 25-30° C. and stirred for 13 hr at the same temperature. Distilled off the solvent from the reaction mixture under reduced pressure. Water (150 ml) and THF (30 ml) were added to the obtained compound at 25-30° C. Cooled the reaction mixture to 0-5° C. and acidified with aq.HCl solution. Raised the temperature of the reaction mixture to 25-30° C. and stirred for 90 min at the same temperature. Filtered the solid, washed with water. MTBE (300 ml) was added to the compound at 25-30° C. and stirred for 90 min at the same temperature. Filtered the solid, washed with MTBE and dried to get title compound. Yield: 27 gm; HPLC Purity: 98.50%.

### Example—10: Purification of Compound of Formula-2

DCM (400 ml) was added to compound of formula-2 (50 gm; having 1.73% of compound of formula-6 as an impurity by HPLC) at 25-30° C. and stirred the mixture for 30 min at the same temperature. Diisopropyl ether (800 ml) was slowly added to the mixture at 25-30° C. and stirred for 2 hr at the same temperature. Filtered the solid, washed with diisopropyl ether and dried to get the title compound. Yield: 42 gm; Purity by HPLC: 99.27%; Histidine impurity (compound of formula-6): 0.01%.

### Example—11: Preparation of Semaglutide Side Chain (Compound of Formula-3a)

### Step 1: Preparation of Compound of Formula-9

DIPEA (64 ml) was slowly added to a mixture of compound of formula-8 (170 gm), acetonitrile (850 ml) and TSTU (110.5 gm) at 25-30° C. and stirred the mixture for 1 hr at the same temperature. Water (1700 ml) was slowly added to the reaction mixture at 25-30° C. and stirred for 90 min at the same temperature. Filtered the solid and washed with water to get the title compound. Yield: 250 gm.

### Step 2: Preparation of Compound of Formula-11

Ethanol (850 ml), DIPEA (64 ml) and 2-(2-(2-aminoethoxy)ethoxy) acetic acid compound of formula-10 (59.8 gm) were added to the compound obtained in step 1 at 25-30° C. and stirred the reaction mixture for 1 hr at the same temperature. Distilled off the solvent from the reaction mixture under reduced pressure. Ethyl acetate and aqueous HCl solution were added to the obtained compound at 25-30° C. Both the organic and aqueous layers were separated and washed the organic layer with aqueous NaCl solution and dried with sodium sulfate. Distilled off the solvent from the organic layer under reduced pressure and co-distilled with DCM to get the title compound. Yield: 204 gm.

### Step 3: Preparation of Compound of Formula-12

Acetonitrile (850 ml), TSTU (92.08 gm) and DIPEA (64 ml) were added to the compound obtained in step 2 at 25-30° C. and stirred the reaction mixture for 1 hr at the same temperature. DIPEA (16 ml) was added to the reaction mixture at 25-30° C. and stirred for 2 hr at the same temperature. TSTU (18.41 gm) was added to the reaction mixture at 25-30° C. and stirred for 30 min at the same temperature to get the title compound.

### Step 4: Preparation of Compound of Formula-13

The reaction mixture obtained in step 3 was slowly added to a mixture of ethanol (850 ml), DIPEA (64 ml) and 2-(2-(2-aminoethoxy)ethoxy) acetic acid compound of formula-10 (50 gm) at 25-30° C. and stirred the obtained reaction mixture for 45 min at the same temperature. Distilled off the solvent from the reaction mixture under reduced pressure. THF, ethyl acetate and aqueous sodium carbonate solution were added to the reaction mixture at 25-30° C. Acidified the reaction mixture by using aqueous HCl solution. Both the organic and aqueous layers were separated and washed the organic layer with aqueous HCl solution followed by with aqueous NaCl solution and dried with sodium sulfate. Distilled off the solvent from the organic layer under reduced pressure and co-distilled with DCM to get the title compound. Yield: 230 gm; Purity by HPLC: 77.92%.

### Step 5: Purification of Compound of Formula-13

The crude compound of formula-13 obtained in step 4 (12 gm) was dissolved in 50% methanol in water at 25-30° C. and sonicated. Stabilized the column with mobile phase A. Loaded the compound solution into reverse phase C18 column and stabilized the column with mobile phase A. Purification cycle was performed by using following conditions; Mobile Phase A: Water (pH adjusted with 0.1% TFA solution); Mobile Phase B: Acetonitrile; Elution: Gradient.

Desired fractions were collected and distilled off the solvent from the fractions. Cooled the compound to 10-15° C. and acidified by using 1N HCl solution. DCM was added to the obtained solution at 10-15° C. Both the organic and aqueous layers were separated. DCM and 5% NaCl solution were added to the aqueous layer and stirred for 20 min. Both the organic and aqueous layers were separated. Combined the organic layers and distilled off the solvent to get the pure compound of formula-13. Yield: 6 gm; HPLC Purity: 99.20%.

### Step 6: Preparation of Compound of Formula-15a

p-Nitro phenol compound of formula-14a (0.9 gm) and HOBt (79 mg) were added to a solution of compound of formula-13 obtained in step 5 (5 gm) in DCM (25 ml) at 25-30° C. and stirred the reaction mixture for 20 min at the same temperature. A solution of DCC (1.58 gm) in DCM (25 ml) was added to the reaction mixture at 25-30° C. DIPEA (1.34 ml) was slowly added to the reaction mixture at 25-30° C. and stirred for 3 hr at the same temperature. Cooled the reaction mixture to 0-5° C. and stirred for 30 min at the same temperature. Filtered the reaction mixture and washed with DCM to get the title compound.

### Step 7: Preparation of Compound of Formula-3a

Water (50 ml) was added to the filtrate obtained in step 6 at 10-15° C. Acidified the reaction mixture by using aq. HCl solution. Raised the temperature of the reaction mixture to 25-30° C. Both the organic and aqueous layers were separated and washed the organic layer with water. TFA (25 ml) was added to the organic layer at 25-30° C. and stirred the reaction mixture for 1 hr at the same temperature. Distilled off the solvent from the reaction mixture under reduced pressure and co-distilled with toluene followed by with MTBE. MTBE (100 ml) was added to the obtained compound at 25-30° C. and stirred for 1 hr at the same temperature. Filtered the solid, washed with MTBE. Diisopropyl ether (50 ml) was added to the obtained compound at 25-30° C. and stirred for 1 hr at the same temperature. Filtered the solid, washed with diisopropyl ether and dried to get the title compound. Yield: 4.1 gm.

### Example—12: Preparation of Compound of Formula-17

DMAP (77.69 gm) and t-butanol (400 ml) were added to a mixture of toluene (3600 ml) and compound of formula-16 (400 gm) at 25-30° C. and stirred the reaction mixture for 30 min at the same temperature. Heated the reaction mixture to 75-80° C. and stirred for 30 min at the same temperature. A solution of DIBOC (222.08 gm) in toluene (1.2 lit) was slowly added to the reaction mixture at 75-80° C. and stirred for 3 hr at the same temperature. A solution of DIBOC (111.04 gm) in toluene (800 ml)) was slowly added to the reaction mixture at 75-80° C. and stirred for 9 hr at the same temperature. Cooled the reaction mixture to 25-30° C., further cooled to 10-15° C. and stirred for 45 min at the same temperature. Filtered the reaction mixture and washed with toluene. Toluene was added to the filtrate at 25-30° C. Cooled the mixture to 5-10° C. and stirred for 45 min at the same temperature. Filtered the reaction mixture and washed with toluene. Distilled off the solvent from the filtrate under reduced pressure. DCM and water were added to the obtained compound at 25-30° C. Cooled the reaction mixture to 5-10° C. and aqueous acetic acid solution was slowly added to it. Raised the temperature of the reaction mixture to 25-30° C. and stirred for 15 min at the same temperature. Both the organic and aqueous layers were separated and washed the organic layer with water. Distilled off the solvent from the organic layer under reduced pressure and co-distilled with n-heptane. n-Heptane (2.8 Lt) was added to the obtained compound at 25-30° C. and stirred for 15 min at the same temperature. Heated the reaction mixture to 65-70° C. and stirred for 15 min at the same temperature. Cooled the reaction mixture to 25-30° C., further cooled to 0-5° C. and stirred for 2 hr at the same temperature. Filtered the solid, washed with n-heptane and dried. IPA (1200 ml) was added to the obtained compound at 25-30° C. and stirred for 15 min at the same temperature. Heated the reaction mixture to 50-55° C., water (1200 ml) was slowly added to it and stirred for 15 mi at the same temperature. Cooled the reaction mixture to 25-30° C. and stirred for 2 hr at same temperature. Filtered the solid, washed with water and dried. IPA (800 ml) was added to the obtained compound at 25-30° C. and stirred for 1 hr at the same temperature. Water (800 ml) was slowly added to the mixture at 25-30° C. and stirred for 2 hr at the same temperature. Filtered the solid, washed with water and dried to get the title compound. Yield: 157.2 gm.

### Example—13: Preparation of Compound of Formula-8

### Step 1: Preparation of Compound of Formula-18

THF (750 ml) was added to compound of formula-17 (150 gm) at 25-30° C. and stirred for 15 min at the same temperature. TSTU (146.2 gm) was added to the reaction mixture at 25-30° C. and stirred for 15 min at the same temperature. DIEPA (84.8 ml) was added to the reaction mixture at 25-30° C. and stirred for 4 hr at the same temperature. DMF (150 ml) was added to the reaction mixture at 25-30° C. and stirred for 3 hr at the same temperature. Distilled off the solvent from the reaction mixture under reduced pressure and co-distilled with acetonitrile. Acetonitrile (750 ml) was added to the obtained compound at 25-30° C. Cooled the mixture to 0-5° C. and stirred for 90 min at the same temperature. Filtered the solid, washed with chilled acetonitrile to get the title compound.

### Step 2: Preparation of Compound of Formula-8

Methanol (750 ml) and compound of formula-19 (82.26 gm) were added to compound obtained in step 1 at 25-30° C. DIPEA (84.8 ml) was slowly added to the reaction mixture at 25-30° C. and stirred the reaction mixture for 1 hr at the same temperature. Distilled off the solvent from the reaction mixture under reduced pressure. Ethyl acetate and aqueous NaHSO₄ solution were added to the obtained compound at 25-30° C. and stirred the mixture for 1 hr at the same temperature. Both the organic and aqueous layers were separated. Distilled off the solvent from the organic layer under reduced pressure and co-distilled with n-heptane. n-Heptane (1500 ml) was added to the obtained compound at 25-30° C. and stirred for 10 min at the same temperature. Cooled the mixture to 0-5° C. and stirred for 5 hr at the same temperature. Filtered the solid, washed with n-heptane and dried to get the title compound. Yield: 197.2 gm; Purity by HPLC: 89.70%.

### Example—14: Preparation of Resin Bound Semaglutide Backbone

### Step 1: Preparation of Fmoc-Gly-CTC Resin

A solution of Fmoc-Gly-OH (9.51 gm) in DCM (175 ml), DMF (25 ml) and DIPEA (11.17 ml) was added to CTC Resin (25 gm; swelled in DCM (200 ml)) at 25-30° C. and stirred the reaction mixture for 4 hr at the same temperature. Drained out the solvent from the peptide flask and washed the reaction mixture with DMF followed by DCM. Capped the resin by using DCM (200 ml), methanol (25 ml) and DIPEA (6.98 ml) at 25-30° C. and stirred the reaction mixture for 45 min at the same temperature. Drained out the solvent from the peptide flask and washed the reaction mixture with DMF to get the title compound.

### Step 2: Deprotection of Fmoc-Gly-CTC Resin

15% Piperidine solution (26.25 ml of piperidine in 148.75 ml of DMF) was added to Fmoc-Gly-CTC Resin obtained in step 1 at 25-30° C. and stirred the reaction mixture for 40 min at the same temperature. Drained out the solvent from the peptide flask and washed the reaction mixture with DMF followed by DCM to get H-Gly-CTC Resin.

Step 3: Preparation of H-Glu (OtBu)-Gly-Thr (tBu)-Phe-Thr (tBu)-Ser (tBu)-Asp (OtBu)-Val-Ser (tBu)-Ser (tBu)-Tyr (tBu)-Leu-Glu (OtBu)-Gly-Gln (Trt)-Ala-Ala-Lys (Boc)-Glu (OtBu)-Phe-Ile-Ala-Trp (Boc)-Leu-Val-Arg (Pbf)-Gly-Arg (Pbf)-Gly-Resin (3-31)

Fmoc-Arg (Pbf)-OH (18 gm) was dissolved in DMF (125 ml) at 25-30° C. HOBt (3.74 gm) and DIC (4.34 ml) were added to the reaction mixture at 25-30° C. and stirred the reaction mixture for 15 min at the same temperature. The resulting solution was added to H-Gly-CTC Resin obtained in step 2 at 25-30° C. and stirred the reaction mixture for 4 hr at the same temperature. Drained out the solvent from the peptide flask and washed the reaction mixture with DMF to get Fmoc-Arg (Pbf)-Gly-CTC Resin. 15% Piperidine solution (26.25 ml of piperidine in 148.75 ml of DMF) was added to the obtained reaction mixture at 25-30° C. and stirred for 40 min at the same temperature. Drained out the solvent from the peptide flask and washed the reaction mixture with DMF to get H-Arg (Pbf)-Gly-CTC Resin.

Repeated the above amino acids coupling and Fmoc deprotection steps with remaining amino acids as per the amino acid sequence of Semaglutide by using Fmoc-Gly-OH, Fmoc-Arg (pbf)-OH, Fmoc-Val-OH, Fmoc-Leu-OH, Fmoc-Trp (Boc)-OH, Fmoc-Ala-OH, Fmoc-Ile-OH, Fmoc-Phe-OH, Fmoc-Glu (OtBu)-OH, Fmoc-Lys (Boc)-OH, Fmoc-Ala-OH, Fmoc-Ala-OH, Fmoc-Gln (Trt)-OH, Fmoc-Gly-OH, Fmoc-Glu (OtBu)-OH, Fmoc-Leu-OH, Fmoc-Tyr (tBu)-OH, Fmoc-Ser (tBu)-OH, Fmoc-Ser (tBu)-OH, Fmoc-Val-OH, Fmoc-Asp (OtBu)-OH, Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH coupled to H-Arg (Pbf)-Gly-CTC resin by linear approach to get the title compound. In case of Fmoc-Glu (OtBu)-OH, Fmoc-Lys (Boc)-OH and Fmoc-Gln (Trt)-OH lot wise addition was performed.

### Step 4: Preparation of Resin Bound Semaglutide Backbone

Boc-His (Trt)-Aib-OH compound of formula-2 (26.93 gm) was dissolved in NMP (150 ml) and DMF (100 ml) at 25-30° C. and HOBt (6.23 gm), DIC (7.23 ml) and DIPEA (4.84 ml) were added to the reaction mixture at the same temperature. This obtained mixture was slowly added lot wise to the compound obtained in step-3 at 25-30° C. and stirred for 24 hr at the same temperature. Drained out the solvent from the peptide flask and washed with NMP followed by methanol and dried to get the title compound. Yield: 70.0 gm.

### Example—15: Cleavage and Global Deprotection to Prepare Semaglutide Backbone

Resin bound Semaglutide backbone (50 gm) was slowly added to a pre-cooled mixture of TFA (332 ml), TIS (20 ml), phenol (40 ml) and water (8 ml) at 10-15° C. and stirred the reaction mixture for 15 min at the same temperature. Raised the temperature of the reaction mixture to 25-30° C. and stirred for 3 hr at the same temperature. Filtered the reaction mixture and washed with TFA. The obtained filtrate was slowly added to pre-cooled MTBE (1500 ml) at 10-15° C. and stirred the reaction mixture for 10 min at the same temperature. Raised the temperature of the reaction mixture to 25-30° C. and stirred for 3 hr at the same temperature. Filtered the solid, washed with MTBE and suck dried. MTBE (1000 ml) was added to the obtained compound at 25-30° C. and stirred the mixture for 2 hr at the same temperature. Filtered the solid, washed with MTBE and suck dried. MTBE (500 ml) was added to the obtained compound at 25-30° C. and stirred the mixture for 2 hr at the same temperature. Filtered the solid, washed with MTBE and dried to get the title compound. Yield: 24.5 gm; Purity by HPLC: 41.43%.

### Example—16: Purification of Semaglutide Backbone by RP-HPLC

Step 1: The crude Semaglutide backbone (100 gm) was dissolved in 1% of aqueous ammonia solution and sonicated. The solution was filtered through 0.45μ filter paper and washed with 1% of aqueous ammonia solution. Equilibrated the column with aqueous Ammonium acetate+aqueous Ammonia. The crude solution was loaded onto the column and the column was stabilized with aqueous Ammonium acetate+aqueous Ammonia. A purification cycle was performed under the following conditions;
Mobile Phase A: Aqueous Ammonium acetate+Aqueous Ammonia; Mobile Phase B: Acetonitrile: Methanol; Elution: Gradient.
Purity by HPLC of the desired fractions: 90.18%.
The desired fractions were collected and distilled off the solvent under reduced pressure to obtain an aqueous solution containing Semaglutide backbone.
Step 2: The C18 column was washed with acetonitrile and 0.1% TFA solution. The solution obtained in step 1 was loaded on to C18 HPLC column and the column was washed with aqueous TFA and followed by equilibrated with aqueous TFA. A purification cycle was performed by using Mobile Phase A: Aqueous TFA and Mobile Phase B: Acetonitrile. Elution: Gradient.
Purity by HPLC of the desired fractions: 95.81%.
The desired fractions were collected and distilled off the solvent from the fractions under reduced pressure and further basified with 5.0% aqueous ammonia solution to obtain an aqueous solution containing Semaglutide backbone.
Step 3: The C18 column was washed with Aqueous Ammonium acetate and then equilibrated with aqueous ammonium acetate. The solution obtained in step 2 was loaded on to the column and stabilized the column with aqueous sodium bicarbonate. The column was subjected to gradient program by using Mobile Phase A: Aqueous TFA solution with pH 4-5; and Mobile phase B: Acetonitrile. Desired fractions were collected and distilled off the solvent from the fractions to obtain an aqueous solution containing Semaglutide backbone. The obtained solution was lyophilized to get pure Semaglutide backbone. Yield: 15 gm; Purity by HPLC: 96.48%.

### Example—17: Preparation of Semaglutide

Water (500 ml) was added to purified Semaglutide backbone (10 gm) at 25-30° C. Cooled the reaction mixture to 15-20° C., DIPEA (5.14 ml) was added to it and stirred the reaction mixture for 10 min at the same temperature. p-Nitro phenol (1.21 gm) and HOBt (0.39 gm) were added to reaction mixture at same temperature. A solution of compound of formula-3a (4.45 gm) in THF (450 ml) was slowly added lot wise to the reaction mixture. DIPEA (8.82 ml) was slowly added to the reaction mixture at 15-20° C. and stirred for 5 hr at same temperature. A solution of compound of formula-3a (0.49 gm) in THF (50 ml) and DIPEA (1.26 ml) were added to the reaction mixture at 15-20° C. and stirred the reaction mixture for 3 hr at the same temperature. Raised the temperature of the reaction mixture to 25-30° C. Ethyl acetate was added to reaction mixture and stirred for 15 min at same temperature. Both the organic and aqueous layers were separated. Ethyl acetate was added to the aqueous layer and stirred for 15 min at same temperature. THF was added to reaction mixture and stirred for 15 min at same temperature. Both the organic and aqueous layers were separated. NaCl was added to the aqueous layer. Cooled the reaction mixture to 10-15° C., slowly acidified with aqueous HCl solution. Raised the reaction mixture temperature to 25-30° C. and stirred for 18 hr at same temperature. Filtered the solid and washed with acetonitrile and suck dried. Acetonitrile (70 ml) was added to the solid at 25-30° C. and stirred for 2 hr. Filtered the solid, washed with acetonitrile and suck dried. Acetonitrile (70 ml) was added to the solid at 25-30° C. and stirred for 2 hr. Filtered the solid, washed with acetonitrile and dried to get the Semaglutide. Yield: 8.42 gm; Purity by HPLC: 89.12%.

### Example—18: Purification of Semaglutide by RP-HPLC

Step 1: The crude Semaglutide (60 gm) was dissolved in 1% aqueous ammonia solution and sonicated. The solution was filtered through 0.45μ filter paper. The C18 column was equilibrated with aqueous sulfuric acid. The crude solution was loaded on to the column and the column was stabilized with aqueous sulfuric acid. A purification cycle was performed by using the following conditions; Mobile Phase A: Aqueous sulfuric acid; Mobile Phase B: Acetonitrile: Aqueous sulfuric acid; Elution: Gradient.
Purity by HPLC of the desired fractions: 98.65%.
The desired fractions were collected and distilled off the solvent under reduced pressure and further 1% aqueous ammonia solution was added to obtain an aqueous solution containing Semaglutide.
Step 2: The C18 column was washed and equilibrated with aqueous ammonium acetate solution. The solution obtained in step 1 was loaded on the column. The column was eluted with gradient program by using Mobile Phase A: aqueous ammonium acetate solution and Mobile Phase B: Acetonitrile: Isopropanol; Purity by HPLC of desired fractions: 99.51%.
The desired fractions were collected and distilled off the solvent under reduced pressure to obtain an aqueous solution containing Semaglutide.
Step 3: C18 column was washed and equilibrated with aq. NaHCO₃ solution. The solution obtained in step 2 was loaded on the column. The column was eluted with gradient program by using Mobile Phase A: Water and Mobile Phase B: Acetonitrile. Desired fractions were collected and distilled off the solvent under reduced pressure. The solution was subjected to lyophilization to get pure Semaglutide as solid. Yield: 36 gm; Purity by HPLC: 99.31%.

### Example—19: Preparation of Compound of Formula-2

### Step 1: Preparation of Compound of Formula-5a

Thionyl chloride (17.6 ml) was slowly added to a mixture of ethanol (102 ml) and 2-aminoisobutyric acid compound of formula-4 (10.2 gm) at 25-30° C. Heated the reaction mixture to 70-75° C. and stirred for 9 hr at the same temperature. Cooled to 50-55° C. Distilled off the solvent from the reaction mixture under reduced pressure at 50-55° C. and co-distilled with ethyl acetate. Ethyl acetate (102 ml) was added to the obtained compound at 25-30° C. and stirred the mixture for 90 min at the same temperature. Filtered the solid, washed with ethyl acetate to get the title compound.

### Step 2: Preparation of Compound of Formula-7a

Tetrahydrofuran (150 ml) was added to compound obtained in step-1 and stirred for 15 min at 25-30° C. Cooled the mixture to 0-5° C., EDC.HCl (17.45 gm) and HOBt (8.14 gm) were added to it and stirred for 15 min at the same temperature. DIPEA (10.5 ml) and Boc-His (Trt)-OH compound of formula-6 (30 gm) were slowly added to the reaction mixture at 0-5° C. DIPEA (10.5 ml) was slowly added to the reaction mixture at 0-5° C. Raised the temperature of the reaction mixture to 25-30° C. and stirred for 7 hr at the same temperature. Cooled the reaction mixture to 0-5° C., water (300 ml) was slowly added to it. Raised the temperature of the reaction mixture to 25-30° C. and stirred for 2 hr at the same temperature. Filtered the solid, washed with water and dried to get the title compound.

### Step 3: Preparation of Compound of Formula-2

THF (210 ml) was added to the compound obtained in step-2 at 25-30° C. Aqueous LiOH solution (3.6 gm of LiOH in 90 ml of water) was added to the reaction mixture at 25-30° C. and stirred for 11 hr at the same temperature. Distilled off the solvent from the reaction mixture under reduced pressure. Water (150 ml) and THF (30 ml) were added to the obtained compound at 25-30° C. Cooled the reaction mixture to 0-5° C. and acidified with aq.HCl solution. Raised the temperature of the reaction mixture to 25-30° C. and stirred for 1 hr 40 min at the same temperature. Filtered the solid, washed with water. MTBE (300 ml) was added to the compound at 25-30° C. and stirred for 1 hr 30 min at the same temperature.

Filtered the solid, washed with MTBE and dried. DCM (240 ml) was added to obtained compound at 25-30° C. and stirred the mixture for 50 min at the same temperature. Diisopropyl ether (480 ml) was slowly added to the mixture at 25-30° C. and stirred for 3 hr at the same temperature. Filtered the solid, washed with diisopropyl ether and dried to get the title compound. Yield: 26 gm; Purity by HPLC: 98.69%; Histidine impurity (compound of formula-6): 0.03%.

### Example—20: Preparation of Semaglutide Side Chain (Compound of Formula-3a)

### Step 1: Preparation of Compound of Formula-9

DIPEA (11.3 ml) and TSTU (19.52 gm) were added to a mixture of compound of formula-8 (30 gm) and acetonitrile (180 ml) at 25-30° C. and stirred the mixture for 2 hr at the same temperature. Water (360 ml) was slowly added to the reaction mixture at 25-30° C. and stirred for 2 hr at the same temperature. Filtered the solid and washed with water to get the title compound.

### Step 2: Preparation of Compound of Formula-11

Ethanol (150 ml), DIPEA (11.3 ml) and 2-(2-(2-aminoethoxy)ethoxy) acetic acid compound of formula-10 (10.58 gm) were added to the compound obtained in step 1 at 25-30° C. and stirred the reaction mixture for 2 hr at the same temperature. Distilled off the solvent from the reaction mixture under reduced pressure. Ethyl acetate and aqueous HCl solution were added to the obtained compound at 25-30° C. and stirred for 1 hr at same temperature. Both the organic and aqueous layers were separated and washed the organic layer with aqueous NaCl solution. Distilled off the solvent from the organic layer under reduced pressure and co-distilled with DCM to get the title compound.

### Step 3: Preparation of Compound of Formula-12

Acetonitrile (150 ml) was added to compound obtained in step 2 at 25-30° C. and stirred for 10 min. TSTU (24.78 gm) and DIPEA (18.87 ml) were added to the reaction mixture at 25-30° C. and stirred for 2 hr at the same temperature to get the title compound.

### Step 4: Preparation of Compound of Formula-13

The reaction mixture obtained in step 3 was slowly added to a mixture of ethanol (150 ml), DIPEA (11.68 ml) and 2-(2-(2-aminoethoxy)ethoxy) acetic acid compound of formula-10 (9.23 gm) at 25-30° C. and stirred the reaction mixture for 90 min at the same temperature. Distilled off the solvent from the reaction mixture under reduced pressure. THE, ethyl acetate and aqueous sodium carbonate solution were added to the reaction mixture at 25-30° C. Acidified the reaction mixture by using aqueous HCl solution and stirred for 1 hr. Both the organic and aqueous layers were separated and washed the organic layer with aqueous NaCl solution. Distilled off the solvent from the organic layer under reduced pressure and co-distilled with DCM to get the title compound. Yield: 40.5 gm; Purity by HPLC: 78.70%.

### Step 5: Purification of Compound of Formula-13

The crude compound of formula-13 (72 gm) was dissolved in 50% methanol in water at 25-30° C. and sonicated. The solution was filtered through 0.45μ filter paper. Washed the column with mobile phase A. Loaded the compound solution into reverse phase C18 column. Equilibrate the column with mobile phase A. Purification cycle was performed by using following conditions; Mobile Phase A: Water (pH adjusted to 3-4 with 0.1% TFA solution); Mobile Phase B: Acetonitrile; Elution: Gradient.

Desired fractions were collected and distilled off the solvent from the fractions. Cooled the compound to 5-10° C. and acidified by using 1N HCl solution and stirred for 15 min at same temperature. DCM was added to the obtained solution at 5-10° C. Both the organic and aqueous layers were separated. DCM and 5% NaCl solution were added to the aqueous layer and stirred for 15 min. Both the organic and aqueous layers were separated. Combined the organic layers and distilled off the solvent to get the pure compound of formula-13. Yield: 41 gm; HPLC Purity: 98.29%.

### Step 6: Preparation of Compound of Formula-15a

p-Nitro phenol compound of formula-14a (4.9 gm) and HOBt (0.935 gm) were added to a solution of compound of formula-13 (25 gm) in DCM (125 ml) at 25-30° C. A solution of DCC (8.97 gm) in DCM (125 ml) was added to the reaction mixture at 25-30° C. DIPEA (7.6 ml) was slowly added to the reaction mixture at 25-30° C. and stirred for 1 hr at the same temperature. Filtered the reaction mixture and washed with DCM. Water was added to the filtrate, acidified with aq. HCl solution and stirred for 10 min at same temperature. Both the organic and aqueous layers were separated and washed the organic layer with water. Dried the organic layer over sodium sulfate to get the title compound.

### Step 7: Preparation of Compound of Formula-3a

TFA (55 ml) was added to the organic layer obtained in step-6 at 25-30° C. and stirred the reaction mixture for 1 hr 50 min at the same temperature. Distilled off the solvent from the reaction mixture under reduced pressure and co-distilled with toluene followed by with MTBE. MTBE (220 ml) was added to the obtained compound at 25-30° C. and stirred for 2 hr at the same temperature. Filtered the solid, washed with MTBE. Diisopropyl ether (110 ml) was added to the obtained compound at 25-30° C. and stirred for 2 hr at the same temperature. Filtered the solid, washed with diisopropyl ether and dried to get the title compound. Yield: 10.18 gm.

### Example—21: Preparation of Compound of Formula-17

DMAP (25.24 gm) and t-butanol (130 ml) were added to a mixture of toluene (1170 ml) and compound of formula-16 (130 gm) at 25-30° C. Heated the reaction mixture to 75-80° C. and stirred for 30 min at the same temperature. A solution of DIBOC (72.17 gm) in toluene (390 ml) was slowly added to the reaction mixture at 75-80° C. and stirred for 3 hr at the same temperature. A solution of DIBOC (36.08 gm) in toluene (260 ml) was slowly added to the reaction mixture at 75-80° C. and stirred for 9 hr at the same temperature. Cooled the reaction mixture to 0-5° C. and stirred for 40 min at the same temperature. Filtered the reaction mixture and washed with toluene. Distilled of the solvent from the filtrate under reduced pressure. Toluene (650 ml) was added to the obtained compound at 25-30° C. Cooled the mixture to 5-10° C. and stirred for 45 min at the same temperature. Filtered the mixture and washed with toluene. Distilled off the solvent from the filtrate under reduced pressure. DCM was added to the obtained compound at 25-30° C. Cooled the mixture to 5-10° C., water followed by aqueous acetic acid solution were slowly added to it. Raised the temperature of the reaction mixture to 25-30° C. and stirred for 15 min at the same temperature. Both the organic and aqueous layers were separated and washed the organic layer with water. Distilled off the solvent from the organic layer under reduced pressure and co-distilled with n-heptane. n-Heptane (910 ml) was added to the obtained compound at 25-30° C. Heated the mixture to 65-70° C. and stirred for 15 min at the same temperature. Cooled the mixture to 0-5° C. and stirred for 1 hr. Filtered the solid, washed with n-heptane and dried. IPA (390 ml) was added to the obtained compound at 25-30° C. and stirred for 15 min at the same temperature. Heated the reaction mixture to 50-55° C. and stirred for 45 min. Water (390 ml) was slowly added to the mixture at 50-55° C. Cooled the mixture to 25-30° C. and stirred for 2 hr at same temperature. Filtered the solid, washed with water and dried. IPA (260 ml) was added to the obtained compound at 25-30° C. and stirred for 15 min at the same temperature. Water (260 ml) was slowly added to the mixture at 25-30° C. and stirred for 2 hr at the same temperature. Filtered the solid, washed with water and dried. IPA (390 ml) was added to the compound at 25-30° C. and stirred for 15 min at the same temperature. Heated the reaction mixture to 50-55° C. and stirred for 15 min. Cooled the reaction mixture to 25-30° C., water (195 ml) was slowly added to it and stirred for 2 hr at the same temperature. Filtered the solid, washed with water and dried to get title compound. Yield: 42.0 gm; Purity by HPLC: 89.41%.

### Example—22: Preparation of Compound of Formula-8

### Step 1: Preparation of Compound of Formula-18

THF (125 ml) was added to compound of formula-17 (25 gm) at 25-30° C. TSTU (24.37 gm) and DIPEA (14.14 ml) were added to the reaction mixture at 25-30° C. and stirred for 4 hr at same temperature. DMF (25 ml) was added to the reaction mixture at 25-30° C. and stirred for 2 hr at same temperature. Distilled of the solvent from the reaction mixture under reduced pressure and co distilled with acetonitrile. Acetonitrile (125 ml) was added to the obtained compound at 25-30° C. Cooled the mixture to 0-5° C. and stirred for 60 min at the same temperature. Filtered the solid, washed with chilled acetonitrile to get title compound.

### Step 2: Preparation of Compound of Formula-8

Methanol (125 ml) and compound of formula-19 (13.71 gm) were added to compound obtained in step 1 at 25-30° C. DIPEA (14.14 ml) was slowly added to the reaction mixture at 25-30° C. and stirred for 3 hr at the same temperature. Distilled off the solvent from the reaction mixture under reduced pressure. Ethyl acetate and aq. NaHSO₄ solution were added to the obtained compound at 25-30° C. and stirred the reaction mixture for 30 min at the same temperature. Both the organic and aqueous layers were separated. Distilled off the solvent from the organic layer under reduced pressure and co-distilled with n-heptane. n-Heptane (250 ml) was added to the obtained compound at 25-30° C. Heated the reaction mixture to 40-45° C. and stirred for 15 min at same temperature. Cooled the mixture to 0-5° C. and stirred for 8 hr at the same temperature. Filtered the solid, washed with n-heptane and dried to get title compound. Yield: 30.6 gm; Purity by HPLC: 92.96%.

### Example—23: Preparation of Compound of Formula-10

### Step 1: Preparation of Compound of Formula-22

2-2-(Chloroethoxy) ethanol (64.21 ml; compound of formula-21), sodium carbonate (53.73 gm) and potassium iodide (8.41 gm) were added to a mixture of dibenzylamine (100 gm; compound of formula-20) and DMF (200 ml) at 25-30° C. Heated the reaction mixture to 120-125° C. and stirred for 10 hr at same temperature. Cooled the reaction mixture 15-20° C. and water was added to it at the same temperature. Acidified the reaction mixture with aqueous HCl solution. MTBE was added to reaction mixture and stirred for 15 min at same temperature. Both the organic and aqueous layers were separated. Aqueous layer was basified with aqueous ammonia solution at 20-25° C. and toluene was added to it. Raised the temperature of the reaction mixture to 25-30° C. and stirred for 15 min at same temperature. Both the organic and aqueous layers were separated and washed the organic layer with water. Distilled off the solvent from the organic layer under reduced pressure to get title compound.

### Step 2: Preparation of Compound of Formula-24

THF (500 ml) was added to the compound obtained in step 1 at 25-30° C. and further cooled to 15-20° C. Potassium tert-butoxide (KOBt; 102.24 gm) was slowly added lot wise to the reaction mixture at 15-20° C. and stirred for 30 min at same temperature. Raised the temperature of the reaction mixture to 25-30° C. and stirred for 2 hr at same temperature. Sodium monochloro acetate compound of formula-23a (106.12 gm) was added lot wise to the reaction mixture at 25-30° C. and stirred for 8 hr at the same temperature. Cooled the reaction mixture to 15-20° C. and water was added to it. MTBE was added to reaction mixture at 15-20° C. and stirred for 15 min at same temperature. Both the organic and aqueous layers were separated and acidified the aqueous layer with aq. HCl solution. DCM was added to the reaction mixture at 15-20° C. Raised the temperature of the reaction mixture to 25-30° C. Both the organic and aqueous layers were separated and extracted the aqueous layer with DCM. Combined the organic layers and triethylamine was added to it at 25-30° C. Distilled off the solvent from the reaction mixture under reduced pressure. MTBE was added to the obtained compound at 25-30° C. and stirred the mixture for 20 min at the same temperature. Charcoal was added to the mixture at 25-30° C. and stirred for 30 min at same temperature. Filtered the reaction mixture, washed with MTBE. Distilled off the solvent from the filtrate to get the title compound.

### Step 3: Preparation of Compound of Formula-25

Methanol (800 ml) was added to the compound obtained in step 2 at 25-30° C. and stirred the mixture for 15 min at same temperature. 5% Pd—C(10 gm) was added to the reaction mixture at 25-30° C. Hydrogen gas pressure was applied to the reaction mixture at 25-30° C. Heated the reaction mixture to 50-55° C. and stirred for 8 hr at same temperature. Cooled the reaction mixture to 25-30° C. Filtered the reaction mixture through hyflow bed and washed the hyflow bed with methanol to get the title compound.

### Step 4: Preparation of Compound of Formula-10

5% Pd—C(10 gm) was added to the filtrate obtained in step 3 at 25-30° C. H₂ gas pressure was applied to the reaction mixture at 25-30° C. Heated the reaction mixture to 50-55° C. and stirred for 9 hr at same temperature. Filtered the reaction mixture through hyflow bed and washed with methanol. Distilled off the solvent from the reaction mixture under reduced pressure. DCM (400 ml) was added to the obtained compound at 25-30° C. Heated the mixture to 45-50° C. and distilled off the solvent under reduced pressure. Isopropanol (200 ml) was added to the obtained compound at 25-30° C. Heated the mixture to 60-65° C. and stirred for 15 min at same temperature. Cooled the mixture to 25-30° C. and stirred 10 hr at same temperature. Filtered the solid, washed with isopropanol and suck dried. Isopropanol (200 ml) and methanol (20 ml) were added to the obtained compound at 25-30° C. Heated the mixture to 60-65° C. and stirred for 45 min at same temperature. Cooled the mixture to 25-30° C. and stirred for 2 hr at same temperature. Filtered the solid, washed with isopropanol and dried to get the title compound. Yield: 42 gm; Purity by HPLC: 99.66%.

1. An improved process for the preparation of Semaglutide, comprising reacting (3-31) amino acid fragment of Semaglutide having the amino acid sequence Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly with PG₁-His (PG)-Aib-OH to provide a peptide having amino acid sequence His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly and optionally converting the obtained compound to Semaglutide, wherein, ‘PG’ and ‘PG₁’ independently represents protecting groups.

2. The process according to claim 1, wherein ‘PG₁’ represents a protecting group selected from alkyloxy carbonyl such as methoxy carbonyl, ethoxy carbonyl, tert.butoxycarbonyl (Boc); benzyloxycarbonyl (Cbz), 9-fluorenylmethyloxy carbonyl (FMOC), acetyl (Ac), benzoyl (Bz), benzyl (Bn), allyloxy carbonyl (Alloc), trityl (Trt); and ‘PG’ represents a protecting group selected from trityl (Trt), tert.butyl (t-Bu), Tosyl (Tos), monomethoxy trityl (Mtt), methyltrityl (Mmt), tert.butoxycarbonyl (Boc), 2,4-dimethylpent-3-yloxycarbonyl (Doc), benzyloxymethyl (Bom), tert-butoxymethyl (Bum).

3. The process according to claim 1, wherein various amino acids of (3-31) amino acid fragment of Semaglutide are optionally protected with different protecting groups which include alkyloxy carbonyl such as methoxy carbonyl, ethoxy carbonyl, tert.butoxy carbonyl (Boc); benzyloxycarbonyl (Cbz), 9-fluorenylmethyloxy carbonyl (FMOC), acetyl (Ac), benzoyl (Bz), benzyl (Bn), allyloxy carbonyl trityl (Trt), tert butyl (tBu), 2,2,4,6,7-pentamethyl-2,3-dihydrobenzofuran-5-sulfonyl (Pbf), Tosyl (Tos), monomethoxy trityl (Mtt), methyltrityl (Mmt), 2,4-dimethylpent-3-yloxycarbonyl (Doc), benzyloxymethyl (Bom), tert-butoxymethyl (Bum).

4. The process according to claim 1, wherein the (3-31) amino acid fragment of Semaglutide is synthesized by solid phase peptide synthesis (SPPS) and is bound to a solid support (Resin).

5. The process according to claim 4, wherein the Resin bound (3-31) amino acid fragment of Semaglutide has the formula Glu-Gly-Thr-Phe-Thr-Ser-Asp-Vai-Ser-Ser-Tyr-Leu-Glu-Gly-Gin-Ala-Ala-Lys-Glu-Phe-Ile-Ala-Trp-Leu-Val-Arg-Gly-Arg-Gly-Resin and the amino acids of the sequence are optionally protected with protecting group(s).

6. The process according to claims 1 and 4, wherein the resulting peptide bound to resin has the amino acid sequence having amino acid sequence His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly and the amino acids are optionally protected with protecting group(s).

7. The process according to claim 5, wherein the Resin is Chlorotrityl chloride (CTC) Resin.

8. The process according to claim 1, wherein the reaction is carried out in a solvent optionally in presence of a coupling agent and/or a base.

9. The process according to claim 8, wherein the solvent is selected from hydrocarbon solvents, ether solvents, ester solvents, polar-aprotic solvents, chloro solvents, ketone solvents, nitrile solvents, water and the like or mixtures thereof, the base is selected from organic bases and the coupling agent is selected from N,N′-dicyclohexylcarbodiimide (DCC), N,N″-diisopropylcarbodiimide (DIC), 1-ethyl-3-(3-dimethylaminopropyl) carbodiimide hydrochloride (EDC.HCl), N,N″-carbonyldiimidazole (CDI), 1-[bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphat (HBTU), 1H-benzotriazolium 1-[bis(dimethylamino)methylene]-5-chloro-hexafluorophosphate (1)-3-oxide (HCTU), (benzotriazol-1-yloxy)tris(dimethyl amino)phosphonium hexafluoro phosphate (BOP), benzotriazol-1-yl-oxytripyrrolidino phosphonium hexafluorophosphate (PyBOP), 1-hydroxy-7-azatriazole, 1-hydroxy benzotriazole (HOBt), 1-hydroxy-1H-1,2,3-triazole-4-carboxylate, O-(benzotriazol-1-yl)-N,N,N′,N′-tetramethyl uranium tetrafluoroborate, N-hydroxysuccinamide, N-hydroxysulfo succinimide, ethyl cyanohydroxyiminoacetate, 7-azabenzotriazol-1-yloxy)tripyrrolidinophosphonium hexafluorophosphate, N,N,N′,N′-Tetramethyl-O-(N-succinimidyl) uronium tetrafluoroborate (TSTU) or mixtures thereof.

10. The process according to claim 1, wherein the conversion of a peptide having amino acid sequence His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly to Semaglutide is carried out by global deprotection of the compound to cleave the peptide chain from the Resin and deprotection of the protected amino acids by using “cocktail mixture/cleaving reagent” and reacting the obtained compound with compound of formula-3 in a solvent optionally in presence of a base and/or a coupling agent. 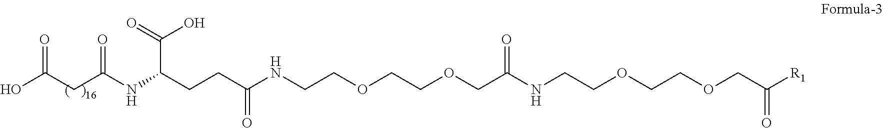
wherein, ‘R₁’ represents substituted or unsubstituted aryloxy and the substituents wherever necessary can be independently selected from halogens such as F, Cl, Br, I; NO₂ and the substitution can takes place at single or multiple positions on aryl group or R₁ is

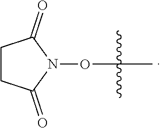 [SMILES: *ON1C(=O)CCC1=O]

11. The process according to claim 10, wherein the “cocktail mixture/cleaving reagent” is selected from HF, TFA (trifluoroacetic acid), TIS or TIPS (triisopropylsilane), phenol, water, anisole, thioanisole, EDT (Ethane-1,2-dithiol), 1-dodecanethiol (DDT), Dithiothreitol (DTT), methanesulfonic acid or mixtures thereof.

12. (canceled)

13. (canceled)

14. The process according to claim 10, wherein Semaglutide obtained can be purified by RP-HPLC comprising aqueous H₂SO₄ as mobile phase.

15. The process according to claim 14, further comprising acetonitrile optionally in mixture with aqueous H₂SO₄ as mobile phase B.

16. (canceled)

17. (canceled)

18. (canceled)

19. (canceled)

20. (canceled)

21. (canceled)

22. (canceled)

23. (canceled)

24. (canceled)

25. The process according to claim 10, wherein the compound of formula-3 is prepared by a process comprising;
a) treating compound of formula-13 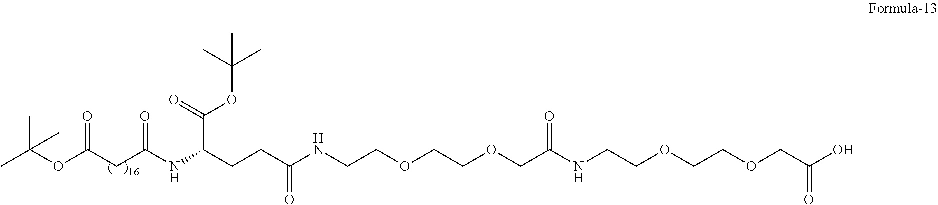
with a compound of formula-14, 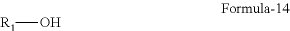
Wherein, R₁′ is as defined above,
in a solvent optionally in presence of a coupling agent and/or a base to provide compound of formula-15, 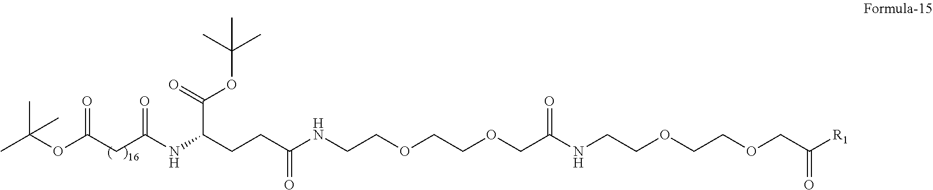
b) treating compound of formula-15 with a deprotecting agent optionally in presence of a solvent to provide compound of formula-3.

26. The process according to claim 25, wherein the compound of formula-13 is prepared by a process comprising reacting compound of formula-11 with compound of formula-10 in a solvent optionally in presence of a coupling agent and/or a base. [chemical structure]
